(19) United States
(12) Patent Application Publication
(10) Pub. No.: US 2026/0218117 A1
(43) Pub. Date: Jul. 30, 2026

(54) ENGINEERED GUT BACTERIA

(71) Applicant: The University of British Columbia, Vancouver (CA)

(72) Inventors: Carolina TROPINI, Vancouver (CA); Juan Camilo BURCKHARDT, Vancouver (CA); Giselle MCCALLUM, Vancouver (CA); Katharine M. NG, Vancouver (CA); Deanna PEPIN, Vancouver (CA)

(21) Appl. No.: 19/373,403

(22) Filed: Oct. 29, 2025

Related U.S. Application Data
(60) Provisional application No. 63/713,207, filed on Oct. 29, 2024.

(51) Int. Cl. C12N 1/205; A61K 35/741; C07K 14/195; C12N 9/14; C12N 15/74; C12R 1/01

(52) U.S. Cl. CPC C12N 1/205; A61K 35/741; C07K 14/195; C12N 9/14; C12N 15/74; C12Y 306/00; C12N 2800/101; C12N 2830/34; C12R 2001/01

(57) ABSTRACT

A VANISH genetically engineered bacterium, a method of making the VANISH genetically engineered bacterium, and pharmaceutical compositions comprising the VANISH genetically engineered bacterium are disclosed. The VANISH genetically engineered bacterium comprises an engineered nucleic acid which comprises a non-native copy of an operon. The operon encodes a betaine transport/import system. In some embodiments, the operon is endogenous to the genome of a bacterial species from the Bacteroidaceae family. In some embodiments, the operon comprises an opuA operon comprising the genes opuAA, opuAB and opuAC which encode an ATPase, a transmembrane protein channel and a periplasmic substrate-binding protein. The VANISH genetically engineered bacterium may be introduced into a mammalian gut to reintroduce gut microbes that are vanishing in industrialized mammalian populations. Introduction of such VANISH genetically engineered bacterium may advantageously provide protection against osmotic stress or other stress response genes to enhance survival in an industrialized mammalian gut.

### CROSS-REFERENCE TO RELATED APPLICATIONS

This application claims the benefit under 35 U.S.C. § 119 of U.S. application No. 63/713,207 filed 29 Oct. 2024 and entitled ENGINEERED GUT BACTERIA.

### SEQUENCE LISTING

This application contains a sequence listing which has been filed electronically in XML format and is hereby incorporated herein by reference in its entirety. The XML file containing the sequence listing was created on 29 Oct. 2025, is named U0081371_Seq-List.xml and is 95,957 bytes in size.

### FIELD

This invention pertains to genetically engineered bacteria, in particular, those that are capable of treating, ameliorating and/or preventing disorders associated with high osmolality or malabsorption in the gut.

### BACKGROUND

The transition of humans from traditional foraging and gathering to industrialized lifestyles led to many changes that modified the gut environment, such as increased sanitization, antibiotic use, and dietary changes. These disruptions have resulted in drastic shifts in the intestinal microbial composition, noticeable since early life in the development of children raised in industrialized societies compared to rural ones. The identification of VANISH (Volatile and/or Associated Negatively with Industrialized Societies of Humans) taxa and their association with lower rates of modern diseases such as obesity, asthma, and inflammatory conditions has spurred an interest in the reintroduction of missing species into the industrialized microbiota. This reintroduction harnesses the functional gene repertoires of these vanishing microbes, reestablishing lost metabolic capabilities, promoting interbacterial symbiotic interactions, and ultimately has the goal of transforming the microbiota landscape to benefit host gut physiology and immunity. However, unless these missing taxa can survive the industrialized human gut habitat, the reintroduction of these species alone will not be sufficient to ensure their maintenance in industrialized societies. Beyond specific nutritional requirements that could, in principle, be introduced as supplements in the diet, the industrialized human intestinal environment is routinely exposed to changes in its physical conditions due to modern diseases or the consumption of drugs that alter factors such as acidity, temperature, or molecule concentration. The latter factor, referred to as osmolality, is commonly increased in the industrialized human gut compared to the gut of humans living in rural environments due to the high prevalence of malabsorption in inflammatory bowel diseases, food intolerances, and the high use of over-the-counter laxatives in industrialized human populations. Unabsorbed molecules in the gut cavity increase intestinal osmolality, leading to the passive outflow of water from epithelial cells into the gut lumen, increased gut motility and decreased stool consistency. These osmotic changes not only affect the host digestive organs, but also have a large impact on the gut microbiota. Notably, osmotic changes exert a strong selection pressure on intestinal microbial communities in mouse models and humans.

The inventors have recognized a general desire and need for compositions and methods for reinstating vanishing gut microbes in industrialized populations.

### SUMMARY

This application has a number of aspects. These include, without limitation:
a VANISH genetically engineered bacterium comprising an engineered nucleic acid comprising a non-native copy of an operon which encodes a betaine transport/import system;
methods of making the VANISH genetically engineered bacterium from conjugation of a plasmid carrying a gene or operon that is native to a genome of the Bacteroides genus into a genome of a bacterium from the VANISH taxa;
pharmaceutical compositions comprising the VANISH genetically engineered bacterium.

In some embodiment of the invention, the VANISH genetically engineered bacterium comprises an engineered nucleic acid which comprises a non-native copy of an operon which encodes a betaine transport/import system. The non-native copy of the operon is operatively linked to a promoter. In some embodiments, the non-native copy of the operon is endogenous to the genome of a bacterial species from the Bacteroidaceae family, and in some embodiments, from the Bacteroides genus.

In some embodiments, the non-native copy of the operon comprises an opuA operon. The opuA operon comprises the genes opuAA, opuAB and opuAC which encode an ATPase, a transmembrane protein channel and a periplasmic substrate-binding protein.

In some embodiments, the non-native copy of the operon comprises a nucleic acid sequence that has at least about 70% sequence identity to the nucleic acid sequence set forth in SEQ IQ NO: 1.

In some embodiments, the promoter comprises a nucleic acid sequence that is native to the genome of a bacterial species from the Bacteroides genus. In some embodiments, the promoter comprises a nucleic acid sequence that is derived from a nucleic acid sequence that is native to the genome of a bacterial species from the Bacteroides genus. In some example embodiments, the nucleic acid sequence of the promoter comprises the -7/-10 consensus sequence of TAnnTTTG. The promoter may in some embodiments comprise a nucleic acid sequence that has at least about 70% sequence identity to the nucleic acid sequence set forth in SEQ IQ NO: 2. The promoter may in some embodiments comprise a nucleic acid sequence that has at least about 70% sequence identity to the nucleic acid sequence set forth in SEQ IQ NO: 35.

In some embodiments, the promoter is an inducible promoter. In some example embodiments, the inducible promoter has a nucleic acid sequence set forth in SEQ IQ NO: 35. In some embodiments, the promoter is a constitutive promoter. In some example embodiments, the constitutive promoter has a nucleic acid sequence set forth in SEQ IQ NO: 2.

The engineered nucleic acid is integrated into a genome of the VANISH bacterium to genetically engineer it. In some embodiments, the engineered nucleic acid is integrated into the genome of the VANISH bacterium at one or more recombinase recognition sites. The one or more recombinase recognition sites may comprise an attBT site. In some embodiments, the attBT site is located in the 3′ end of a serine tRNA (Ser-tRNA) gene.

In some embodiments, the VANISH bacterium is from one of Muribaculaceae, Prevotellaceae, Spirochaetaceae and Succinivibrionaceae families.

Aspects of the invention pertain to use of the VANISH genetically engineered bacterium. In some embodiments, the VANISH genetically engineered bacterium is used in treating, ameliorating and/or preventing disorders associated with high osmolality or malabsorption in a gut of a mammal.

Further aspects and example embodiments are illustrated in the accompanying drawings and/or described in the following description.

It is emphasized that the invention relates to all combinations of the above features, even if these are recited in different claims.

### BRIEF DESCRIPTION OF THE DRAWINGS

The accompanying drawings illustrate non-limiting example embodiments of the invention.

FIG. 1 is a schematic diagram illustrating an expression construct contained in a VANISH genetically engineered bacterium according to an example embodiment of the invention. The expression construct comprises a promoter that is operatively linked to a non-native operon which encodes a betaine transport/import system.

FIGS. 2A to 2F illustrate the results pertaining to the experiments described in Example 1. The FIGS. 2A to 2F results demonstrate that overexpression of the B. thetaiotaomicron (Bt) opuA operon in M. intestinale NM06 (Mi NM06) increases its osmotic resilience in vitro. FIG. 2A is a schematic diagram illustrating a genome alignment of homologous attBT integration sites found in serine tRNA genes in Bt, Mi and Mi NM06. FIG. 2B is a graph showing the aligned promoter regions of Bt, Mi, and Mi NM06 which contain the Bacteroides-7/-10 consensus sequence. FIG. 2C illustrates graphs showing the results of fluorescence quantification of Bt and Mi NM06 cultures driving GFP expression from the engineered Bt BT1311 promoter which comprises a sequence set forth in SEQ ID NO: 2. FIG. 2D illustrates graphs showing the CFU/mL of Mi NM06 opuA⁺⁺ and opuA^{P-less} strains grown in vitro in media with different levels of PEG at different timepoints. FIG. 2E is a graph showing the maximum OD₆₀₀ of Mi NM06 opuA⁺⁺ and opuA^{P-less} in minimal media with increasing osmolality due to PEG with or without added 2 mM betaine, and FIG. 2F is a graph showing the growth rate measurements of Mi NM06 opuA⁺⁺ and opuA^{P-less} in minimal media with increasing osmolality due to PEG with or without added 2 mM betaine. L.O.D: limit of detection.

FIGS. 3A to 3E illustrate the results pertaining to the experiments described in Example 2. The FIGS. 3A-3E results demonstrate that overexpression of opuA protects Mi NM06 from laxative-induced osmotic stress in the gut. FIG. 3A illustrates an experimental diagram illustrating a mouse experiment with low PEG dosages (5-7%). FIG. 3B is a graph showing fecal density of Mi NM06 opuA^{P-less} or opuA⁺⁺ quantified via absolute qPCR. FIG. 3C is a graph showing cecal osmolality measured at D12 indicates low levels of malabsorption. FIG. 3D is an experimental diagram illustrating a mouse experiment with high PEG dosages (10-15%). FIG. 3E is a graph showing fecal density of Mi NM06 opuA^{P-less} or opuA⁺⁺ quantified via absolute qPCR.

FIGS. 4A to 4C illustrate the results pertaining to the experiments described in Example 3. The FIGS. 4A-4C results demonstrate that betaine addition rescues Mi NM06 opuA⁺⁺ osmotic tolerance during high laxative dosages. FIG. 4A is an experimental diagram illustrating a mouse experiment with high PEG dosage and added betaine. FIG. 4B is a graph showing fecal density of Mi NM06 opuA^{P-less} or opuA⁺⁺ quantified via absolute qPCR. FIG. 4C is a graph showing cecal osmolality measured at day 7 of PEG treatment.

FIG. 5 illustrates results which demonstrate that betaine presence reduces the lag phase of Mi NM06 opuA⁺⁺ at high osmolalites. FIG. 5 is a graph showing the lag phase extracted from Gompertz curve fitting of Mi NM06 opuA⁺⁺ and opuA^{P-less} growth curves done in minimal media at varying osmolalities with and without 2 mM betaine. NA: not available/no growth.

FIG. 6A is a schematic diagram illustrating an expression construct with an inducible promoter that is operatively linked to the non-native opuA operon as used in the experiments described in Example 4.

FIG. 6B is a schematic diagram illustrating an expression construct with an inducible promoter that is operatively linked to sfGFP as used in the experiments described in Example 4.

FIG. 7 are plots of CFU/mL as a function of time of M. intestinale containing the construct from FIG. 6A grown in media with different osmolalities. The FIG. 7 plots show that as more inducer anhydrotetracycline (aTc) is added and expression of opuA is increased, there is a limit past which over expression of the system reduces cell viability.

FIG. 8 is a plot of GFP (AU) as a function of aTc (ng/ml) in M. intestinale containing the construct from FIG. 6B. FIG. 8 illustrates the different levels of expression as measured with the fluorescence output.

FIG. 9 is a plot of (Area under the curve, or AUC below) as a function of AAM Osmolality (mOsm/kg). The results show different levels of survival. In examples of no induction, there is leaky expression but no survival at high osmolality, grey bar. At greater than 12 ng/ml of aTc inducer, the growth is worsened.

### DETAILED DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive sense.

### Definitions

The term “gut” means the gastrointestinal or digestive tract (also referred to as the alimentary canal), and refers to the system of organs within mammals (e.g., humans and animals) which takes in food, digests it to extract energy and nutrients, and expels the remaining waste. A gut that has been “industrialized” or also referred to as an “industrialized gut” is a gut that has been affected by industrialization that may be driven by lifestyle changes.

An “operon” is a functioning unit of genomic DNA containing a cluster of genes under the control of a single promoter.

A “betaine transport system” is a system which imports extracellular betaine, a common osmoprotectant widely found in dietary plants and involved in protection from high osmolality environments in plants and bacteria. An example betaine transport system is one which is encoded by the OpuA operon. The opuA operon comprises the genes opuAA, opuAB and opuAC which encode an ATPase, a transmembrane protein channel and, a periplasmic substrate-binding protein.

A “non-native” or “exogenous” gene/operon refers to a gene/operon that is not normally present in the genome of a bacterium. Conversely, a “native” or “endogenous” gene/operon refers to a gene/operon that is normally present in the genome of the bacterium.

To determine the “sequence identity” of two polynucleotide sequences, the sequences are aligned for optimal comparison purposes (e.g., gaps can be introduced in the sequence of a first polynucleotide for optimal alignment with a second polynucleotide sequence). The nucleotides at corresponding nucleotide positions are then compared. When a position in the first sequence is occupied by the same nucleotide as the corresponding position in the second sequence, then the nucleotides are identical at that position. The sequence identity between the two sequences is a function of the number of identical positions shared by the sequences (i.e., % identity=number of identical positions/total number of positions (i.e., overlapping positions)×100). In some embodiments the length of a reference sequence aligned for comparison purposes is at least 50, 60, 70, or 80% of the length of the comparison sequence, and in some embodiments is at least 90% or 100%. In an embodiment, the two sequences are the same length.

The term “promoter” is a nucleic acid sequence involved in the binding of RNA polymerase to initiate transcription of an operably linked gene. The gene includes a coding DNA sequence and other (non-coding) sequences, e.g., the 5′-untranslated region (5′-UTR) located upstream of the coding sequence, which comprises a ribosomal binding site. Promoters can be constitutive, inducible, repressible, or tissue-specific. Promoters can contain genetic elements at which regulatory proteins and molecules such as RNA polymerase and transcription factors may bind.

The term “operably linked” refers to the expression of a polynucleotide that is under the control of a promoter with which it is spatially connected. A promoter can be positioned 5′ (upstream) of an operon/gene under its control.

The term “repressor” refers to a molecule such as a protein that binds to operator of DNA or to RNA to prevent transcription or translation, respectively. Non-limiting examples of suitable repressor proteins include TetR, LacI, and PhIF.

An “inducible promoter” is a promoter that is characterized by regulating (e.g., initiating or activating) transcriptional activity when in the presence of, influenced by or contacted by an inducer signal. An inducer signal may be endogenous or a normally exogenous condition (e.g., pH, light, temperature, osmotic pressure, saline gradients, and the concentration of one or more extrinsic or intrinsic inducing agent(s) such as ions, salts, toxins enzyme substrate analogs, hormones or combinations thereof), small molecule or compound (e.g., chemical or non-chemical compound) or protein that contacts an inducible promoter in such a way as to be active in regulating transcriptional activity from the inducible promoter. A “signal that regulates transcription” of a nucleic acid refers to an inducer signal that acts on an inducible promoter. A signal that regulates transcription may activate or inactivate transcription, depending on the regulatory system used. Activation of transcription may involve directly acting on a promoter to drive transcription or indirectly acting on a promoter by inactivation a repressor that is preventing the promoter from driving transcription. Deactivation of transcription may involve directly acting on a promoter to prevent transcription or indirectly acting on a promoter by activating a repressor that then acts on the promoter.

A “constitutive promoter” refers to a promoter that is capable of facilitating continuous transcription of a coding sequence or gene under its control and/or to which it is operably linked.

A “pharmaceutical composition” refers to a preparation of genetically engineered bacteria with other components such as a physiologically suitable carrier and/or excipient.

The term “excipient” refers to an inert substance added to a pharmaceutical composition to further facilitate administration of an active ingredient. Calcium bicarbonate, calcium phosphate, various sugars and types of starch, cellulose derivatives, gelatin, vegetable oils, polyethylene glycols, and surfactants, including, for example, polysorbate 20 are non-limiting examples of suitable excipient.

The term “probiotic” is used to refer to live, non-pathogenic microorganisms, e.g., bacteria, which can confer health benefits to a host organism that contains an appropriate amount of the microorganism.

### Example Embodiments

Some aspects of the invention pertain to genetically engineered bacteria that may be introduced into a mammalian (e.g., human) gut to reintroduce gut microbes that are vanishing in industrialized mammalian populations. In some embodiments, an engineered nucleic acid is integrated into a genome of a VANISH (Volatile and/or Associated Negatively with Industrialized Societies of Humans) bacterium to genetically engineer it to form a “VANISH genetically engineered bacterium”. Some non-limiting examples of bacterial families from the VANISH taxa include Muribaculaceae, Prevotellaceae, Spirochaetaceae and Succinivibrionaceae. In some example embodiments, the VANISH bacterium is a bacterium from the Muribaculum genus.

In some embodiments, the VANISH genetically engineered bacteria are stably maintained in the mammalian gut of industrialized populations. In such embodiments, the VANISH genetically engineered bacteria stably produce one or more exogenous proteins that allow them to survive against the common environmental conditions (e.g., high osmolarity) in an industrialized mammalian gut. Some other aspects of the invention pertain to pharmaceutical compositions comprising the VANISH genetically engineered bacteria. In some further aspects, the invention provides a composition comprising the VANISH genetically engineered bacteria for providing protection against osmotic stress or other stress response genes to enhance survival in an industrialized mammalian gut. The VANISH genetically engineered bacteria may for example be formulated as a bacterial probiotic or a live biotherapeutic for use in the treatment of, without limitation, Type-1 diabetes, IBD, celiac disease, dietary intolerances and other disorders related to high gut osmolalities or malabsorption of the gut and enteric pathogen exclusion.

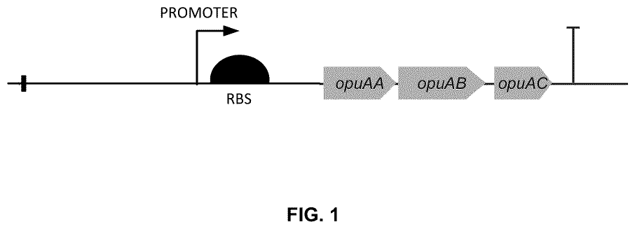

Referring to FIG. 1, in some embodiments, the VANISH genetically engineered bacterium comprises an engineered nucleic acid comprising a non-native or exogenous copy of an operon which encodes a betaine transport/import system.

In some embodiments, the non-native copy of the operon may encode an opuA system. In such embodiments, the operon comprises an opuA operon with three structural genes, opuAA, opuAB and opuAC which encode an ATPase, a transmembrane protein channel, and a periplasmic substrate-binding protein. In some embodiments, the opuA operon comprises a nucleic acid sequence that is endogenous or native to the genome of a bacterial species from the Bacteroidaceae family, and in some embodiments, from the Bacteroides genus. Examples of Bacteroides species and strains include but are not limited to Bacteroides humanifaecis, Bacteroides sp. HF-162, Bacteroides sp. A1C1, sp. CACC 737, stercoris, Bacteroides graminisolvens, Bacteroides xylanisolvens, Bacteroides sp. DH3716P, Bacteroides ovatus, Bacteroides luhongzhouii, Bacteroides sp. M10, Bacteroides sp. D2, Bacteroides finegoldii, Bacteroides zhangwenhongii, Bacteroides caccae, Bacteroides fragilis, Bacteroides sp. PHL 2737, Bacteroides sp. ZJ-18, Bacteroides nordii, Bacteroides salyersiae, Bacteroides thetaiotaomicron, Bacteroides faecis, Bacteroides acidifaciens, Bacteroides caccae, Bacteroides distasonis, Bacteroides gracilis, Bacteroides dorei, Bacteroides oris, Bacteroides putredinis, Bacteroides pyogenes, Bacteroides suis, Bacteroides tectus, Bacteroides vulgatus, Bacteroides eggerthii, Bacteroides merdae, Bacteroides uniformis, etc.

In some example embodiments, the opuA operon comprises a nucleic acid sequence set forth in SEQ ID NO: 1. In some example embodiments, the opuA operon comprises a nucleic acid sequence that has at least about 70%, 80%, 90%, 95%, 99%, or 70%-99%, or 80%-99%, or 90%-99% sequence identity to the nucleic acid sequence set forth in SEQ ID NO: 1.

The non-native copy of an operon which encodes a betaine transport/import system is operatively linked directly or indirectly to a promoter. A nucleotide sequence encoding a ribosome binding site (RBS) may be operably linked to the promoter. In some embodiments, a nucleotide sequence encoding a repressor is operatively linked to the promoter.

A suitable promoter drives the expression of the non-native copy of the operon which encodes the betaine transport/import system in the VANISH genetically engineered bacterium. In some embodiments, a suitable promoter is one that is capable of driving expression of the non-native copy of the operon in the VANISH genetically engineered bacterium at a level that is about 10- to 30-fold, and in some embodiments, about 15- to 25-fold, and in some embodiments, about 20-fold less than the expression of the engineered promoter (SEQ ID NO: 2) driving the opuA operon in Bacteroides, In some embodiments, the promoter is not associated with the operon which encodes the betaine transport/import system.

In some example embodiments, the promoter comprises a Bacteroides promoter. A Bacteroides promoter refers to a promoter sequence that is native or endogenous to the genome of the genus Bacteroides. In some embodiments, the promoter comprises a sequence which is derived from a promoter sequence that is native to the genome of the genus Bacteroides. In some embodiments, a nucleic acid is “derived from” (another) nucleic acid, means that the nucleic acid, which is derived from (another) nucleic acid, shares at least about 50% sequence identity with the nucleic acid from which it is derived.

In some embodiments, the promoter comprises the -7/-10 consensus sequence of TAnnTTTG. In some example embodiments, the promoter is one of BT1311, BT1751, PBfP1E6 or comprises a sequence which is derived therefrom. In some example embodiments, the promoter comprises a BT1311 (rpoD) derived promoter comprising a nucleic acid sequence set forth in SEQ ID NO: 2, or a nucleic acid sequence that has at least about 70%, 80%, 90%, 95%, 99%, or 70%-99%, or 80%-99%, or 90%-99% sequence identity to the nucleic acid sequence set forth in SEQ ID NO: 2.

In some embodiments, the promoter is inducible. In such embodiments, the non-native copy of the operon that encodes the betaine transport system is expressed under the control of an inducible promoter. In some embodiments, the non-native copy of the operon that encodes the betaine transport system is expressed under the control of a promoter that is directly or indirectly induced by an inducer. In some embodiments, the inducer comprises a small molecule such as but is not limited to anhydrotetracycline (aTc), isopropyl β-D-1-thiogalactopyranoside and 2,4-diacetylphloroglucinol. In some example embodiments, the inducible promoter has a nucleic acid sequence set forth in SEQ ID NO: 35, or a nucleic acid sequence that has at least about 70%, 80%, 90%, 95%, 99%, or 70%-99%, or 80%-99%, or 90%-99% sequence identity to the nucleic acid sequence set forth in SEQ ID NO: 35.

In some embodiments, the promoter is constitutive. In some example embodiments, the constitutive promoter may be a weak promoter for sustained expression of the operon, e.g., the opuA operon. In some example embodiments, a suitable constitutive promoter is one which has a maximum expression of about 10%, and in some embodiments, of about 5%.

A nucleic acid sequence encoding a ribosome binding site (RBS) may be operably linked to the promoter. Such nucleotide sequence may be positioned between the promoter and the non-native copy of the operon which encodes the betaine transport/import system. In some embodiments, a ribosome binding site is positioned 3′ of the promoter and 5′ of the nucleotide sequence encoding the non-native copy of the operon which encodes the betaine transport/import system. In some embodiments, the sequence of the ribosome binding site is the sequence between the promoter sequence and the nucleotide sequence encoding the non-native copy of the operon which encodes the betaine transport/import system. A ribosome binding site is a sequence within the mRNA that is bound by the ribosome when initiating protein translation.

In some example embodiments, the nucleic acid sequence which encodes the ribosome binding site is set forth in SEQ ID NO: 3, or has at least about 70%, 80%, 90%, 95%, 99%, or 70%-99%, or 80%-99%, or 90%-99% sequence identity to the nucleic acid sequence set forth in SEQ ID NO: 3.

In some embodiments, the non-native copy of the operon that encodes the betaine transport/import system is expressed on a chromosome. In some embodiments, the non-native copy of the operon that encodes the betaine transport/import system is integrated into the bacterial chromosome at an integration site in a VANISH bacterium.

In some embodiments, the non-native copy of the operon that encodes the betaine transport/import system is integrated into the bacterial genome at one or more recombinase recognition sites such as att sites (e.g., attB, attP, attL, and attR sites), lox sites (e.g., LoxP, Lox511, Lox2272, Lox2372, Lox5171, Loxm2, Lox71, Lox66, LoxFas), and frt sites. In some example embodiments, the integration site comprises one or both of the attBT sites (i.e., attBT-1 and attBT-2) located in the 3′ end of a serine tRNA (Ser-tRNA) gene. Any suitable integration site may however be used. The integration site may however be in other suitable locations within the genome. In some example embodiments, the integration site in the plasmid is located between the integrase and the antibiotic resistance cassette. In some example embodiments, the plasmid is a pNBU2 plasmid.

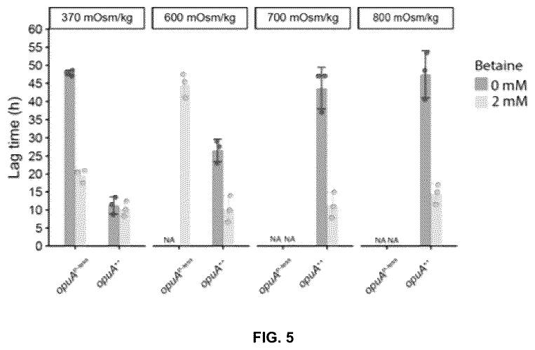

In some embodiments, the VANISH genetically engineered bacterium contains an expression construct which comprises the promoter that is operatively linked to the non-native copy of the operon which encodes a betaine transport/import system. The expression construct may be integrated into the bacterial genome. In some embodiments, the expression construct comprises an integrative plasmid such as a pNBU2, pNBU1, or pExchange plasmid. Other suitable expression constructs may be used. A suitable expression construct may be selected for use in different bacterial chromosomal integration methods (e.g., site-specific recombination systems, homologous recombination, transposons, etc.). An example expression construct is schematically illustrated in FIG. 1. Another example expression construct which comprises an inducible promoter is schematically illustrated in FIG. 6A.

Some aspects of the invention pertain to methods of producing a VANISH genetically engineered bacterium which expresses an operon that is native to the genome of a bacterial species from the Bacteroidaceae family such as a species from the Bacteroides genus. Such operon is hereinafter referred to as “Bacteroides operon”. The operon is non-native to the VANISH genetically engineered bacterium. An expression construct as described herein comprises a promoter that is operatively linked to the Bacteroides operon. The method comprises a conjugation step which comprises mating of an expression construct containing donor strain (e.g., Escherichia coli such as but is not limited to E. coli S17-1 λpir) and a recipient strain from the VANISH taxa to form a mixture. The mating of the donor strain and the recipient strain may be performed by incubating the mixture comprising the two strains under aerobic conditions for a first time interval (e.g., between 6 hours to 48 hours, and in some embodiments, about 24 hours), followed by incubating the strains under anaerobic conditions for a second time interval (e.g., between 6 hours to 6 days, and in some embodiments, about 2 days). In some embodiments, the ratio of donor strain to recipient strain in the mixture is about 1:2 v/v, and in some embodiments between about 1:1 v/v and about 1:4 v/v, and in some embodiments, between about 1:1 v/v and about 1:3 v/v. The mixture may be incubated in a suitable medium. The media conditions (e.g., the composition of the media, and concentration(s) which make up the media) may be selected based on the specific bacterial strains that are being conjugated. Non-limiting examples of media that may be used in the conjugation step are listed below. After the conjugation step, the engineered or conjugated bacteria (i.e., those with a genome which contains the non-native copy of the Bacteroides operon) may be selected. The selection step selects against the donor strain. In some embodiments, the selection step comprises incubating the mixture with media comprising one or more antibiotics (e.g., erythromycin, kanamycin, etc.). In some example embodiments, the genome integration is validated by PCR assay. Example media conditions that may be used in the conjugation step are:
Salyer's minimal medium (MM (or MM-TC for Mi NM06)) which comprises 1 g/L (NH₄)₂SO₄, 1 g/L of Na₂CO₃, 0.5 g/L of cysteine, 100 ml of 1 M KPO₄ (pH 7.2), 1 mL of vitamin K3 solution (1 mg/mL), 10 mL of FeSO₄ solution (0.4 mg/mL), and 0.5 mL of vitamin B12 solution (0.01 mg/mL), 50 mL of mineral salts solution, 2 mL of Hemin solution (5 mg/mL), 5 g/L dextrose. For Mi NM06, add 2.5 g/L of Bactotryptone (Gibco) and 2.5 g/L of Casaminoacids (ThermoScientific).
Tryptone-yeast extract-glucose growth medium (TYG-Agar) which comprises TYG broth supplemented with 1.5% agar.
Supplemented Brain Heart Infusion blood agar (BHIS-BA) which comprises BHI broth supplemented with 1.5% agar, 5% sheep's blood, 5 mg/L hemin solution, and 1 mg/L vitamin K1 solution.
Columbia blood agar (COLB) which comprises Columbia broth supplemented with 1.5% agar, 5% sheep's blood, 5 mg/L hemin solution, and 1 mg/L vitamin K1 solution.
Luria-Bertani agar (LB-agar) which comprises LB broth supplemented with 1.5% agar.
Anaerobic Akkermansia Medium (AAM) which comprises 18.5 g/l brain heart infusion (BHI), 5 g/L yeast extract, 15 g/L trypticase soy broth, 2.5 g/L K₂HPO₄, 1 mg/L haemin, 0.5 g/L glucose, 0.4 g/l Na₂CO₃, 0.5 g/L cysteine hydrochloride, 5 mg/L menadione, 5 mg/L vitamin K1, 3% (v/v) heat shock treated bovine serum albumin (Fisher).
Chopped Meat Medium (CM) from Anaerobe Systems.
5 mg/L hemin solution (Hemin) which comprises 50 mg hemin chloride dissolved in 1 mL 1N NaOH and 9 mL Milli-Q water.
1 mg/L Vitamin K1 solution (Vit. K1) which comprises 100 μL vitamin K1 in 10 mL 95% ethanol.
500 mg/mL Cysteine-HCl solution (Cys-HCl) which comprises 1 g of L-Cysteine Hydrochloride Monohydrate dissolved in 2 mL Milli-Q water.
Mineral salts solution (Mineral salts) which comprises 18 g/L NaCl, 0.53 g/L of CaCl₂·2H₂O, 0.40 g/L of MgCl₂·6H₂O, 0.20 g/L of MnCl₂·4H₂O, 0.20 g/L of CoCl₂·6H₂O.
25 mg/mL erythromycin (Erm) which comprises 250 mg of erythromycin dissolved in 10 mL 95-100% ethanol.
50 mg/mL kanamycin (Kan) which comprises 500 mg of kanamycin dissolved in 10 mL ddH₂O.

Some aspects of the invention pertain to pharmaceutical compositions and formulations comprising the described VANISH genetically engineered bacterium and use thereof to provide protection against osmotic stress or other stress response genes to enhance survival in the mammalian gut of industrialized populations. In some embodiments, such pharmaceutical compositions and formulations are used in the treatment of various disorders and diseases such as Type-1 diabetes, IBD, celiac disease, dietary intolerances and/or other disorders related to high gut osmolalities or malabsorption of the gut and enteric pathogen exclusion.

The pharmaceutical compositions described herein may be formulated in a conventional manner using one or more physiologically acceptable carriers comprising excipients and auxiliaries, which facilitate processing of the active ingredients into compositions for pharmaceutical use. For example, the pharmaceutical composition may include, but is not limited to, the addition of calcium bicarbonate, sodium bicarbonate, calcium phosphate, various sugars and types of starch, cellulose derivatives, gelatin, vegetable oils, polyethylene glycols, and surfactants, including, for example, polysorbate 20. The VANISH genetically engineered bacterium may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

In some embodiments, the pharmaceutical compositions are subjected to tabletting, lyophilizing, direct compression, conventional mixing, dissolving, granulating, levigating, emulsifying, encapsulating, entrapping, or spray drying to form tablets, granulates, nanoparticles, nanocapsules, microcapsules, microtablets, pellets, or powders, which may be enterically coated or uncoated. Appropriate formulation depends on the route of administration.

The VANISH genetically engineered bacterium may be formulated into pharmaceutical compositions in any suitable dosage form (e.g., liquids, capsules, sachet, hard capsules, soft capsules, tablets, enteric coated tablets, suspension powders, granules, or matrix sustained release formations for oral administration) and for any suitable type of administration (e.g., oral, topical, injectable, intravenous, sub-cutaneous, suppository, immediate-release, pulsatile-release, delayed-release, or sustained release).

The invention is further described with reference to the following specific examples, which are not meant to limit the invention, but rather to further illustrate it.

### EXAMPLES

In the following Examples, the opuA operon which encodes a betaine transport/import system that is native to Bacteroides thetaiotaomicron (Bt) from the Bacteroidaceae family was integrated into the genome of Muribaculum intestinale NM06 (Mi NM06) from the Muribaculaceae family to produce a VANISH genetically engineered bacterium according to an example embodiment of the invention.

### Example 1: Expression of Bt OpuA in M. intestinale Renders it Osmotically Tolerant In Vitro

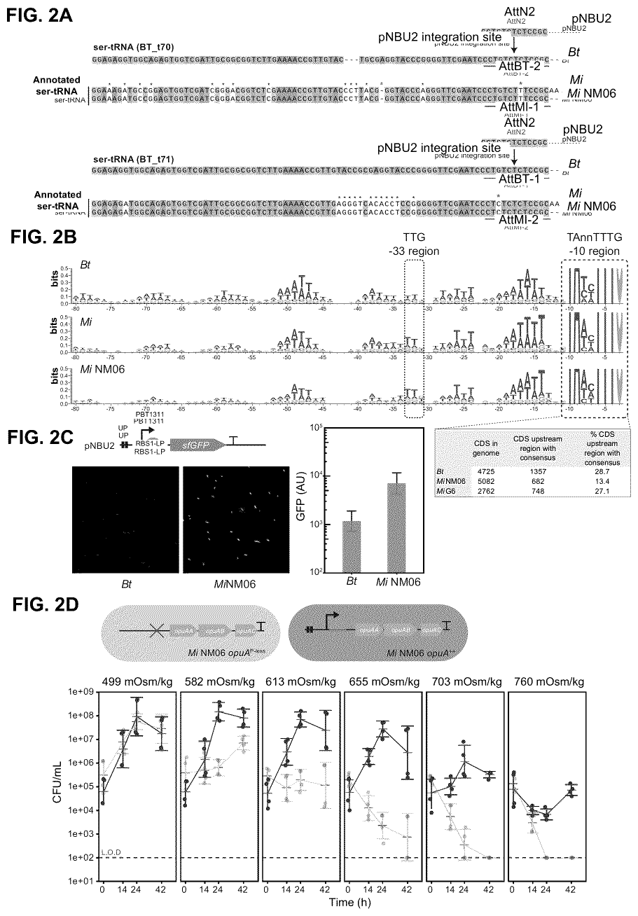

In this Example, the ability of a pNBU2 integrative plasmid (the sequence of which is set forth in SEQ ID NO. 30) to genetically engineer M. intestinale was tested. To test the integration of pNBU2 into M. intestinale strains (human isolate M. intestinale G6 (Mi) as well as the mouse isolate M. intestinale NM06 (Mi NM06)), an engineered sfGFP plasmid construct was conjugated using a customized conjugation protocol tailored to M. intestinale culture requirements as detailed elsewhere herein. The pNBU2 plasmid was integrated into two distinct attBT sites found in the 3′-ends of serine tRNA genes. Post-conjugation, both M. intestinale strains exhibited 79-fold increase in fluorescence levels compared to their WT counterparts, indicating successful conjugation as well as strong expression from an engineered Bt-derived promoter which comprises a nucleic acid sequence set forth in SEQ ID NO. 2 (FIG. 2C). Integration of pNBU2 at the predicted attBT sites was further confirmed by PCR.

The engineered Bt-derived promoter was engineered by replacing the UP element sequence of the strong BT1311 (rpoD) promoter with one from a previously identified strong phage promoter (referred herein as the BT1311UP promoter).

For all subsequent experiments, Mi NM06 strain was used. Next, an opuA⁺⁺ plasmid which comprises a nucleic acid sequence set forth in SEQ ID NO. 30 was conjugated into M. intestinale. As a control, a promoterless version of this construct (opuA^{P-less}) was conjugated SEQ ID NO, 31. To test whether opuA expression would rescue Mi NM06 viability during high osmolality, the opuA^{P-less} and opuA⁺⁺ strains were cultured in complex media at different physiologically relevant osmolalities. PEG was used to adjust the osmolality of the media as it more closely resembles the conditions the strains would encounter during osmotic diarrhea. When grown in baseline osmolality (500 mOsm/kg), Mi NM06 opuA⁺⁺ grew to a similar density as the opuA^{P-less} control (FIG. 2D). However, at elevated osmolalities, the opuA⁺⁺ strain demonstrated a 2- to 5-log increase in carrying capacity compared to the control, with the biggest differences observed at 650 and 700 mOsm/kg (FIG. 2D).

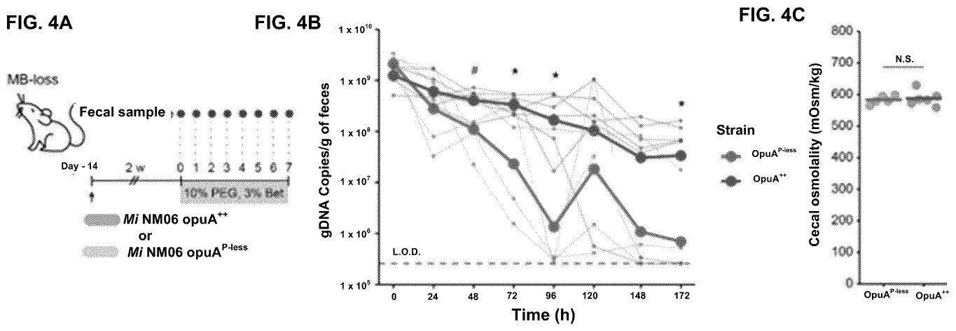

To test that osmotic tolerance conferred by opuA in Mi NM06 may be dependent on the presence of betaine, the inventors performed growth curves with the Mi NM06 opuA^{P-less} and opuA⁺⁺ strains in minimal media, with or without betaine, across a range of osmolalities. At baseline osmolality, both strains had similar carrying capacity and growth rates regardless of betaine supplementation, except for opuA^{P-less} strain grown with betaine, which showed a substantial increase in growth (FIG. 2E-F). Interestingly, at 600 mOsm/kg condition, the opuA⁺⁺ showed similar growth with or without betaine; however, in the absence of betaine, it exhibited a 2.5-fold longer lag phase (FIGS. 2E-F and FIG. 5). This comparable growth may result from trace amounts of betaine or other osmoprotectants present in the tryptone and casamino acid components of the media, enabling the opuA⁺⁺ strain to grow without added betaine. Conversely, at 600 mOsm/kg, the opuA^{P-less} strain grew only when betaine was added, and even then, displayed a lower carrying capacity and growth rate, along with a 4.5-fold longer lag phase relative to the opuA⁺⁺ strain and a 4.5-fold longer lag phase (FIGS. 2E-F and FIG. 5). This limited growth may be due to transcriptional bleed-through of the opuA operon in the strain. At osmolalities exceeding 600 mOsm/kg, growth was observed exclusively in the opuA⁺⁺ strain, with significantly higher carrying capacity and growth rate, and markedly shorter lag phase when betaine was added, suggesting that betaine addition directly enhances osmotic tolerance.

Altogether, these findings support that the opuA operon plays a role in mediating betaine import in Mi NM06, rescuing its growth under osmolality levels reflective of malabsorptive conditions in vitro.

### Example 2: Expression of Bt OpuA in M. intestinale Enables its Survival in a Complex Microbiome Pre-Clinical Model of Laxative-Induced Malabsorption

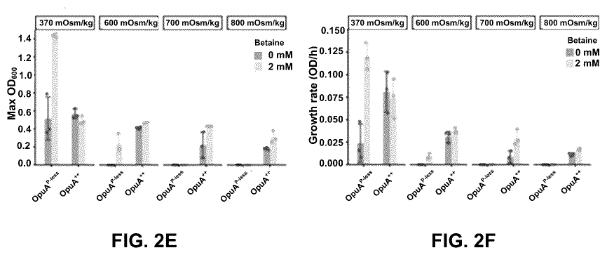

To test whether the opuA⁺⁺ strain enhances Mi NM06 resilience to osmotic challenge in vivo, the inventors colonized mice with a complex microbiota but depleted of MB¹⁸ (MB-free) with either the opuA⁺⁺ or opuA^{P-less} strain. After equilibration, mice were treated with 5% PEG for 6 days, then switched to 7% PEG for an additional 6 days (FIG. 3A). To quantify the strains' colonization levels, the inventors perform absolute qPCR on fecal samples collected before PEG (D0), and at the conclusion of both 5% and 7% PEG treatments (D6 and D12, respectively).

Prior to PEG treatment (D0), both strains reached a density of ~1×10⁹ cells per gram of feces. After 5% PEG treatment (D6), both strains decreased in the fecal density by around 2- to 10-fold, with one mouse colonized with the opuA^{P-less} strain decreasing by about 100-fold (FIG. 3B). By the end of 7% PEG treatment (D12), the opuA⁺⁺ strain maintained an average density of around ~1×10⁸ cells per gram of feces, while the opuA^{P-less} showed a 1,000-fold lower density (~1×10⁶ cell per gram) (FIG. 3B). The final cecal osmolality was around 500 for opuA^{P-less} mice and 520 mOsm/kg for opuA⁺⁺ mice (FIG. 3C), both approximately 100 mOsm/kg higher than baseline cecal osmolality. These preliminary results suggest that opuA expression protects Mi from low levels of malabsorption in the gut.

The inventors then tested if the opuA⁺⁺ osmotic tolerance phenotype would survive at higher dosages of laxatives. MB-free mice were colonized with either the opuA⁺⁺ or opuA^{P-less} strain and treated for 3 days with 10%, 12%, and 15% PEG, consecutively (FIG. 3D). Both strains reached a similar level of colonization compared to the previous experiment (~1×10⁹ cells per gram of feces) (FIG. 3E). After 3 days of 10% PEG treatment, the opuA⁺⁺ strain decreased by approximately 40-fold, whereas the opuA^{P-less} strain declined by approximately 300-fold, indicating a substantial colonization advantage conferred by opuA expression. However, under 12% and 15% PEG treatments, both strains declined to similar levels-approximately 1×10⁶ and 1×10⁴ cells per gram of feces, respectively. This suggests that at higher PEG concentrations, the compounded effects of osmotic stress overwhelmed the protective advantage provided by opuA expression in NM06.

### Example 3: Expression of Bt opuA in M. intestinale NM06 Rescues its Loss During Malabsorption In Vivo During High Laxative Treatment when Betaine is Supplemented

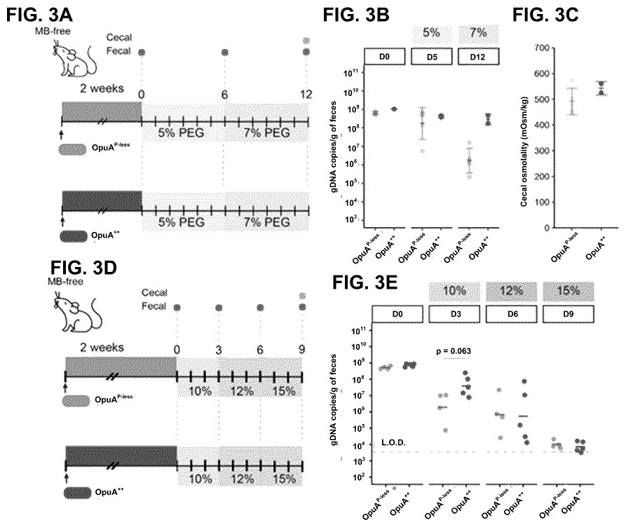

To test whether betaine depletion in the gut lumen limits the protective effect conferred by opuA⁺⁺ on Mi NM06 during high-dose laxative treatment, Mi NM06 opuA⁺⁺ exhibited reduced carrying capacity and growth rate in the absence of betaine (FIG. 2E-F). To test this hypothesis in vivo, MB-free mice were colonized with either Mi NM06 opuA⁺⁺ or opuA^{P-less} and were administered with 10% PEG and 3% betaine to their drinking water for 6 days. Fecal DNA was extracted from daily samples, and strain abundance was quantified via qPCR (FIG. 4A). Both Mi NM06 strains colonized at around 10⁹ copies/g prior to PEG administration (FIG. 4B). Mi NM06 opuA⁺⁺ abundance decreased gradually throughout the treatment, maintaining a final abundance of around 10⁸ copies per grams of feces by the last day of PEG. In contrast, the opuA^{P-less} strain dropped sharply to around 10⁶ copies per grams of feces by 96 h of PEG treatment and remained at low abundance levels near the limit of detection thereafter (FIG. 4B). Final cecal osmolalities were around 590 mOsm/kg for mice colonized with either strain (FIG. 4C), indicating that the observed colonization differences were attributable to betaine supplementation rather than differences in osmotic stress.

Together, these results support that exogenous betaine facilitates opuA⁺⁺-dependent persistence of Mi NM06 during malabsorption, protecting it from luminal depletion. Moreover, the observation that betaine availability controls the persistence of the opuA⁺⁺ strain suggests a potential biocontainment strategy, where dietary modulation could be used to regulate the engraftment of engineered strains in the gut.

### Example 4: PTetR56 Inducible Construct for OpuA Expression

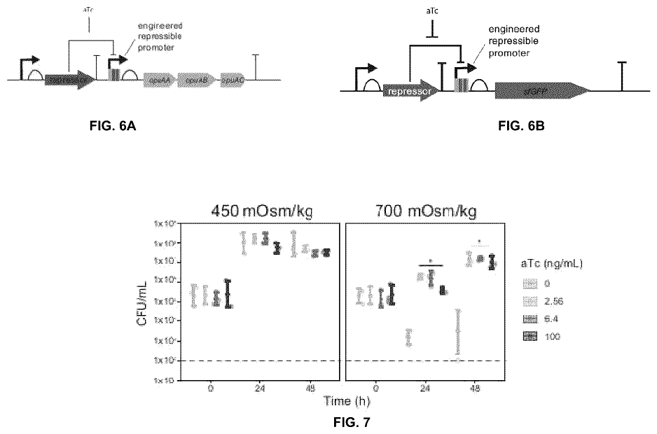

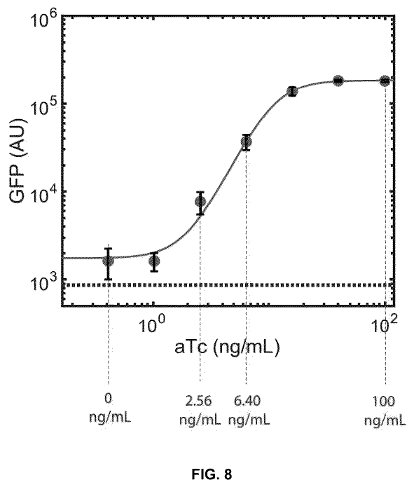

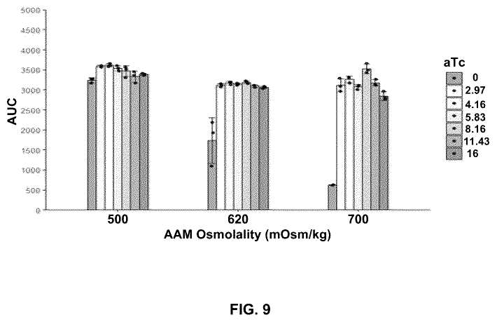

A construct with an inducible promoter for OpuA expression as schematically illustrated in FIG. 6A was tested. The promoter is operatively linked to opuA (FIG. 6A), or GFP (see the FIG. 6B expression construct). The FIG. 7 plots show that if too much inducer anhydrotetracycline (aTc) is added, cell growth is impaired particularly at high osmolality. If no inducer is added, the cells have poor survival at high osmolality. In FIG. 8, the different levels of expression upon addition of inducer are measured with the fluorescence output. The data from FIG. 9 show a measure of growth for a strain harbouring the inducible promoter linked to the OpuA operon and grown in base, medium, and high osmolality with different levels of inducer. The results show different levels of growth depending on opuA induction. In examples of no induction, there is leaky expression but no survival at high osmolality, grey bar. At greater than 12 ng/ml of aTc inducer, the growth is worsened. Based on expression data from FIG. 8, the inventors observed that low levels of opuA (induction with <5 ng/ml aTc; 20-fold less than maximum expression from the construct) are sufficient for growth in medium and high osmolalities. The nucleic acid sequence of the pNBU2 plasmid with the constitutively expressed TetR (driven by the PBPfP2E5 promoter and using the RBS1p-LP Ribosomal binding site), and PTetR56 promoter driving the opuA operon (also using the RBS1p-LP) is set forth in SEQ ID NO: 36 (pJB94).

### Experimental Model and Subject Detail

### Mouse Experiments

Gnotobiotic mice were generated from germ-free (GF) Swiss Webster colonies. The gnotobiotic mice were maintained in gnotobiotic isolators or ISOcage P-Bioexclusion System (Techniplast) under constant high positive pressure and fed an autoclaved standard diet (Purina Lab Diet 5K67) for the duration of the experiments. The mice were sacrificed at various time points using carbon dioxide asphyxiation, followed by secondary euthanasia of cervical dislocation or cardiac puncture.

### Microbial Strains and Growth Conditions

All bacterial isolates used in the experiments were grown in anaerobic conditions (90% N₂, 5% CO₂, 5% H₂) at 37° C. inside a vinyl anaerobic chamber (Coy Laboratory Products) as previously described and detailed below. M. intestinale isolate (human isolate G6) was grown on Columbia agar (BD Difco) supplemented with 5% sheep blood (Dalynn Biologicals™) and chopped meat liquid medium (CM, Anaerobe Systems™ AS-811). M. intestinale NM06, NM06 opuA⁺⁺, and NM06 opuA^{Pless} strains were grown in BHIS agar supplemented with Cysteine-HCl (0.5 g/L), or with Cysteine-HCl (0.5 g/L), erythromycin (25 μg/mL) and kanamycin (50 μg/mL) to select for engineered M. intestinale NM06 strains. M. intestinale NM06 strains were grown in AAM or CM Broth (Anaerobe Systems™).

### Plasmid Construction

Unless indicated elsewhere herein, the plasmids and primers used in the Experiments are listed below. Sequences of plasmids used are listed in the Sequence Listing as SEQ ID NOs: 29 to 34 and 36.

### Plasmids Used in the Experiments

pNBU2-PJ23100-mCherry-PBT1311-sfGFP (pWW3536) which comprises at least the following features: mCherry and sfGFP constitutive expression. pNBU2-ermGb backbone.
pNBU2-PBT1311UP-sfGFP (pEM189) which comprises at least the following features: sfGFP constitutively expressed from PBT1311UP with ribosomal binding site RBS1p-LP, pNBU2-ermGb backbone.
pNBU2-PBT1311UP-opuA (pJBOA) which comprises at least the following features: opuA operon constitutively expressed from PBT1311UP with ribosomal binding site RBS1p-LP, pNBU2-ermGb backbone.
pNBU2-opuA-promoterless (pJB061) which comprises at least the following features: Same as JBOA but with no promoter driving opuA. pNBU2-ermGb backbone.

### Primers Used in the Experiments

The sequences are listed from 5′-3′.
EM351 with the sequence: ctagcgatcgaggtctcaagtc (SEQ ID NO. 4). The amplified product is Assembly PCR primer for PBT1311UP (Outer F). This primer was used to construct the pEM189 plasmid.
EM383 with the sequence: atcgtaggtctccacgggttttattag (SEQ ID NO. 5). The amplified product is Assembly PCR primer for PBT1311UP (Outer R). This primer was used to construct the pEM189 plasmid.
EM388 with the sequence: gtaggtctccacgggttttattagttgttaatatagtgttaaagttgcctaaatatgtatgttaacaaattatttgt cg (SEQ ID NO. 6). The amplified product is Assembly PCR primer for PBT1311UP (Inner F). The primer was used to construct the pEM189 plasmid.
EM384 with the sequence: ctagcgatcgaggtctcaagtcggagtgcaaagttacgacaaataatttgttaacatacatatttaggca a (SEQ ID NO. 7). The amplified product is Assembly PCR primer for PBT1311UP (Inner R). The primer was used to construct the pEM189 plasmid.
pEM189_F with the sequence: gaaagattgaatcctagctgattagctcgagaagg (SEQ ID NO. 8). The amplified product is pNBU2 backbone F. The primer was used to construct the pJBOA plasmid.
pEM189_R with the sequence: caactaataaaacgaattccaattcgaggggg (SEQ ID NO. 9). The amplified product is pNBU2 backbone R. The primer was used to construct the pJBOA plasmid.
opuA_F with the sequence: caataatttattttcaatgagtaaaatagaaattaaagatctctatctggtttttgg (SEQ ID NO. 10). The amplified product is opuA operon F (Bt genome). The primer was used to construct the pJBOA plasmid.
opuA_R with the sequence: tcagctaggattcaatctttctcaggaatccaactgttcac (SEQ ID NO. 11). The amplified product is opuA operon R (Bt genome). The primer was used to construct the pJBOA plasmid.
pBT1311_F with the sequence: gaattggaattcgttttattagttgttaatatagtgttaaagttgcctaaatatgtatgt (SEQ ID NO. 12). The amplified product is pBT1311UP F. The primer was used to construct the pJBOA plasmid.
pBT1311_R with the sequence: ctattttactcattgaaaataaattattgttaatattacctttgaatctcttttcgagtg (SEQ ID NO. 13).
   The amplified product is pBT1311UP R. The primer was used to construct the pJBOA plasmid.
JB131 with the sequence: ggattcctgagaaagattgaatcctagctgat (SEQ ID NO. 14). The amplified product is no_prom_gibson_F. The primer was used to construct the pJB061 plasmid.
JB132 with the sequence: ttaatttctattttactcatgaattccaattcgagggggatcaatt (SEQ ID NO. 15). The amplified product is no_prom_gibson_R. The primer was used to construct the pJB061 plasmid.
JB133 with the sequence: tccccctcgaattggaattcatgagtaaaatagaaattaaagatctctatctggtttttgg (SEQ ID NO. 16). The amplified product is opuA_gibson_F. The primer was used to construct the pJB061 plasmid.
JB134 with the sequence: tcgagctaatcagctaggattcaatotttctc (SEQ ID NO. 17). The amplified product is opuA_gibson_R. The primer was used to construct the pJB061 and pJB094 plasmids.
EM376 with the sequence: cggagtgatgttatgacttacg (SEQ ID NO. 18). The amplified product is Bt genome at attBT1 site.
EM377 with the sequence: ccgactttacattcttgggtg (SEQ ID NO. 19). The amplified product is Bt genome at attBT1/2 site.
EM378 with the sequence: catgtctttatacttattataggctgc (SEQ ID NO. 20). The amplified product is Bt genome at attBT1 site.
EM381 with the sequence: ggaatggatatttttatgatgcgg (SEQ ID NO. 21). The amplified product is Bt genome at attBT2 site.
EM382 with the sequence: cctctatttccagttcacgttg (SEQ ID NO. 22). The amplified product is Bt genome at attBT2 site.
EM377 with the sequence: ccgactttacattcttgggtg (SEQ ID NO. 23). The amplified product is MiNM06 genome at attBT1 site.
EM438 with the sequence: ctattaacatttagcctctacacatcatg (SEQ ID NO. 24). The amplified product is MiNM06 genome at attBT1 site.
EM439 with the sequence: accgggttttaaaagaaggg (SEQ ID NO. 25). The amplified product is MiNM06 genome at attBT1 site.
EM377 with the sequence: ccgactttacattcttgggtg (SEQ ID NO. 26). The amplified product is MiNM06 genome at attBT2 site.
EM440 with the sequence: ggttcgaatccctctctctcc (SEQ ID NO. 27). The amplified product is MiNM06 genome at attBT2 site.
EM441 with the sequence gctogcagagatatccatcag (SEQ ID NO. 28). The amplified product is MiNM06 genome at attBT2 site.
EM427 with the sequence: atcctagctgattagctcgagaagg (SEQ ID NO. 37). The amplified product is F Gibson primer for pNBU2 backbone. The primer was used to construct the pJB094 plasmid.
EM315 with the sequence: tgaaaataaattattgttaatattacctttgaatctc (SEQ ID NO. 38). The amplified product is R Gibson Primer for pNBU2 Backbone and PTetR56 amplification. The primer was used to construct the pJB094 plasmid.
EM316 with the sequence: gattcaaaggtaatattaacaataatttattttcaatgtccagattagataaaagtaaagtgattaacag (SEQ ID NO. 39). The amplified product is F Gibson primer for PTetR56 amplification. The primer was used to construct the pJB094 plasmid.
JB221 with the sequence: aaggtaatattaacaataatttattttcaatgagtaaaatagaaattaaagatctctatctggtttttgg (SEQ ID NO. 40). The amplified product is F Gibson primer for opuA amplification. The primer was used to construct the pJB094 plasmid.
   pWW3536 pNBU2 Plasmid Construction

Using Gibson Assembly, the pWW3536 pNBU2 plasmid was modified to introduce BsaI Golden gate recognition sites immediately upstream of sfGFP. The BT1311 (rpoD) promoter with an altered UP regulatory element (from the strong PBfPE1E6 phage promoter) and a strong Bacteroides RBS (RBS1P-LP) were synthesized using assembly PCR. Briefly, two long primers with 58° C. Tm overhanging regions were annealed and used as templates for PCR. Double-stranded sequences were purified (Qiagen PCR cleanup kit) and inserted into the modified pWW3536 plasmid (pEM189) using Golden Gate assembly. To build an opuA overexpression vector for B. thetaiotaomicron, the sfGFP gene was then replaced with the B. thetaiotaomicron opuA operon: the pEM189 vector excluding the sfGFP gene, as well as the native opuA operon, were amplified with PCR and joined using Gibson Assembly (pJBOA). The resulting pJBOA plasmid was then used as a template to construct the opuA^{P-less} plasmid (JB061) by amplifying the entire vector excluding the PBT1311UP and RBS sequences.

To build the inducible opuA plasmid (pJB094), the sfGFP gene from the existing PTetR56-sfGFP pNBU2 plasmid (FIG. 6B) was replaced with the native opuA operon using PCR and Gibson Assembly.

### Conjugations

The resulting constructs were transformed into E. coli S17-1 λpir cells made chemically competent with CaCl₂) buffer (0.1 M CaCl2/10% glycerol) or TSS buffer (10% w/v Polyethylene glycol 8000, 5% v/v high purity Dimethyl Sulfoxide, 30 mM MgCl2, bring to 50 mL with autoclaved LB) using the Gibson Assembly Transformation Protocol (NEB). Transformants were verified via sanger and full-plasmid nanopore sequencing. Plasmids were introduced into M. intestinale NM06 via conjugation with transformed E. coli S17-1 λpir, described below.

pNBU2 Conjugations into M. Intestinale NM06

A visible loopful of M. intestinale NM06 (recipient) colonies was inoculated into CM media and grown for 3 days until stationary phase (OD 600 nm~0.4). Single colonies of pNBU2 containing E. coli S17-1 λpir (donor) were grown aerobically until stationary phase (OD 600 nm~1.0) one night before M. instestinale NM06 cultures reached stationary phase. The overnight E. coli donor strains were then subcultured at 1:100 and grown aerobically for 4-5 hours. Once the subcultured E. coli donor reached mid-exponential phase (OD 600 nm~0.4-0.5), they were aliquoted into 1 mL microfuge tubes and centrifuged at 4,800×g for 5 minutes to pellet. The pellet was washed twice with fresh CM media, and on the last wash, it was resuspended and concentrated in 100 μL of CM media. The M. intestinale NM06 cultures were aliquoted into 1 mL microfuge tubes, centrifuged at 4,800×g for 5 minutes to pellet, and their supernatant removed. Next, the concentrated E. coli donor was used to resuspend the M. intestinale NM06 pellet (1:1 donor: recipient, v/v), and the entire mixture was spotted onto BHIS with Cysteine-HCl (0.5 g/L) agar plates and incubated aerobically at 37° C. agar-side-up overnight. The following day, the spotted plates were transferred into the anaerobic chamber and incubated agar-side-down anaerobically at 37° C. for 2 days. After anaerobic incubation, a loopful of the growth from the spotted mixture was inoculated into 2 mL of CM media containing added erythromycin (25 μg/mL) and kanamycin (50 μg/mL) to select for conjugated M. intestinale. The selective liquid broth was grown for two nights, and then diluted 1:10 and 1:100 in the same media. 200 μL of each dilution was spotted into BHIS plates containing erythromycin (25 μg/mL) and kanamycin (50 μg/mL) and grown for 2-3 days until single colonies were visible. Single colonies were inoculated into 100 μL of selective CM media with erythromycin (25 μg/mL) and kanamycin (50 μg/mL), grown for 2-3 nights, and then subcultured into larger CM volumes to make glycerol stocks. The single-colony liquid cultures were used to check for integration of pNBU2 in the M. intestinale NM06 genome via colPCR (Figure S7 and Table S6). In Bacteroides, the pNBU2 plasmid integrates into two different attBT sites found in the 3′-end of serine tRNA genes. The inventors identified homologous pNBU2 integration sites (attBT) in Mi NM06, a mouse isolate strain, in annotated serine tRNA genes (FIG. 2A). The putative attBT sites had 100% coverage relative to the Bt attBT sites with only 1 mismatch between the covered regions (FIG. 2A).

### Bacterial Growth Curve Assays-Mi NM06

A loopful of Mi NM06 strains was inoculated into pre-reduced Minimal Medium with added tryptone and casaminoacids (MM-TC) and incubated anaerobically for two night. Cultures were then subcultured 1:40 into MM-TC in a 96-well plate format. MM-TC was supplemented with PEG to varying osmolalities, and betaine was added to half the wells at a final concentration of 2 mM. Three biological replicates were performed for each osmolality +/−betaine condition. OD600 measurements were collected every 5 minutes for 88 hours with 1 minute of orbital shacking between measurements. Sterile blank wells of MM-TC were run, averaged, and subtracted from the OD600 readings of seeded wells. Growth rates were interpreted as previously described using https://github.com/Tropini-lab/Strain library paper.

### In Vitro M. Intestinale NM06 Spot-Plating Quantification

A visible loopful of M. intestinale NM06 opuA⁺⁺ and NM06 opuA^{Pless} colonies was inoculated into AAM with erythromycin (25 μg/mL) liquid media and grown for three days until stable phase was reached. To make AAM osmolality media, AAM was supplemented with PEG to varying levels of osmolality and measured using an Advanced Instruments Osmo1 Single-sample Micro-osmometer. The stable phase cultures were then subcultured 1:10 into the AAM osmolality media conditions and incubated anaerobically at 37° C. until 14, 24, and 42 hours were reached. For every timepoint, 100 μL samples were collected from each condition and diluted anaerobically in PBS by tenfold serial dilutions in a 96-well plate. 5 μL of each dilution was then spot-plated on selective agar plates and incubated at 37° C. until colonies were countable (2-3 days).

M. intestinale NM06 opuA⁺⁺ and opuA^{P-Less} In Vivo Experiments

To produce Muribaculaceae free (MB-free) mice, 4- to 5-week-old male Swiss Webster mice were treated with 15% PEG (w/v) water for 6 days, with daily cage changes to prevent recolonization. Following PEG treatment, mice were recovered for 2 weeks under normal drinking water (for experiments with results illustrated in FIG. 3).

Low laxative dosage experiment: Confirmed MB-free mice were orally gavaged with 200 μL of stable-phase cultures of M. intestinale NM06 opuA⁺⁺ or M. intestinale NM06 opuA^{P-less} grown in either Chopped Meat media or Anaerobic Akkermansia Media (AAM) (OD600~ 0.7). Mice were housed inside sterile biocontainment isocages for the duration of the experiment. After 2-weeks of bacterial equilibration, 5% (w/v) polyethylene glycol (PEG 3350, Miralax™) at a final osmolality of 25 mOsm/kg was supplemented to the drinking water for 6 days. After 6 days, mice were then switched to 7% PEG water at a final osmolality of 40 mOsm/kg. Fecal samples were collected before PEG treatment, and after the conclusion of the 5% and 7% PEG treatments. Fecal samples were flash frozen in dry ice upon collection and stored at −80° C. until used. At the end of the experiment, mice were euthanized, and cecal contents were collected.

### High Laxative Dosage Experiment:

Confirmed MB-free mice were orally gavaged with 200 μL of stable-phase cultures of M. intestinale NM06 opuA⁺⁺ orM. intestinale NM06 opuA^{P-less} grown in Chopped Meat media (OD600~0.4). Mice were housed inside sterile biocontainment isocages for the duration of the experiment. After 2-weeks of bacterial equilibration, 10% PEG 3350 at a final osmolality of 105 mOsm/kg was supplemented to the drinking water for 3 days. After 3 days, mice were then switched to 12% PEG (137 mOsm/kg) and then to 15% PEG water (258 mOsm/kg). Fecal samples were collected before PEG treatment, and after the conclusion of the 10%, 12 and 15% PEG treatments. Fecal samples were flash frozen in dry ice upon collection and stored at −80° C. until used.

High Laxative Dosage with Added Betaine Experiment:

To expand the MB-free mouse colony, cecal contents from confirmed MB-free mice were collected, resuspended in sterile PBS, and 200 μL of the suspension was gavaged into Swiss Webster GF mice (for experiments in FIG. 6). Subsequently, these mice were gavaged with 200 μL of stable-phase cultures ofM. intestinale NM06 opuA⁺⁺ or M. intestinale NM06 opuA^{P-less} grown in Chopped Meat media (OD600~ 0.4). Mice were housed inside sterile biocontainment isocages for the duration of the experiment. After 1-weeks of equilibration, 10% PEG and 3% betaine (w/v; Alfa Aesar) at a final osmolality of 452 mOsm/kg was supplemented to the drinking water for 7 days. Fecal samples were collected prior to PEG treatment, and daily during PEG treatment. Fecal samples were stored at −80° C. until used. At the end of the experiment, mice were euthanized, and cecal contents were collected.

Fecal DNA was extracted from ~30-100 mg samples using the DNeasy PowerSoil Pro Kit (Qiagen). The concentration of extracted DNA was measured with a NanoDrop Lite (Thermo) spectrophotometer. For absolute genome copy number quantification of Mi strains, real-time quantitative PCR (qPCR) was performed with 25 μL reaction volume using PerfeCTa SYBR Green FastMix (Quantabio); 300 nM final primer concentration per well and 20 ng of extracted DNA per well. Thermal cycling was performed in a C1000 Touch Thermal Cycler (Biorad) with the following conditions: initial denaturation of 95° C. for 2 min, 35 cycles of 95° C. for 20 s, 58° C. for 30 s, and 72° C. for 20 s. For standard curves, threshold cycle (Ct) values demonstrated a linear dependence (R2=0.99) to the standard concentration value, and PCR efficiency ranged between 90 and 110%.

Absolute bacterial genome quantification was determined using a standard curve method and the following primers targeting the Ampicillin resistance genes unique to the Mi engineered strains: F: 5′-gttgccattgctacaggcat-3′; R: 5′-ttgcacaacatgggggatca-3′. Standard curves were generated using known amounts of purified bacterial genomic DNA (gDNA) extracted from stable phase cultures of the Mi strains using the DNeasy PowerSoil Pro Kit (Qiagen™). Purified gDNA was quantified via nanodrop and used as stock for a tenfold serial dilution standard curve. Based on the generated calibration curves, the resulting sample Cq values were converted to gDNA copies/g of cecal content.

### Statistical Analysis

For tests on discrete or nominal variables, a Fisher exact test was used with Bonferroni correction to control the family-wise error rate. If two treatments were compared, a Student's T-test for parametric or Two-tailed nonparametric Mann-Whitney U-test for non-parametric data was used. For greater than two treatments, one-way ANOVA with Dunnett's multiple comparisons test, Tukey's multiple comparisons test, or Holm-Sidak's multiple comparisons test were used as appropriate. For non-parametric data, a Mixed-effects model with Geisser-Greenhouse correction was used as appropriate. Mean values are denoted as mean±SD, with significance levels marked as *(p<0.05), **(p<0.01), ***(p<0.001), and ****(p<0.0001). A 0.05>P<0.1 significance value is printed on the corresponding plots and denotes a trend.

### Interpretation of Terms

Unless the context clearly requires otherwise, throughout the description and the claims:
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms. These terms (“a”, “an”, and “the”) mean one or more unless stated otherwise;
“and/or” is used to indicate one or both stated cases may occur, for example A and/or B includes both (A and B) and (A or B);
“approximately” when applied to a numerical value means the numerical value ±10%;
where a feature is described as being “optional” or “optionally” present or described as being present “in some embodiments” it is intended that the present disclosure encompasses embodiments where that feature is present and other embodiments where that feature is not necessarily present and other embodiments where that feature is excluded. Further, where any combination of features is described in this application this statement is intended to serve as antecedent basis for the use of exclusive terminology such as “solely,” “only” and the like in relation to the combination of features as well as the use of “negative” limitation(s)” to exclude the presence of other features; and
“first” and “second” are used for descriptive purposes and cannot be understood as indicating or implying relative importance or indicating the number of indicated technical features.

Where a range for a value is stated, the stated range includes all sub-ranges of the range. It is intended that the statement of a range supports the value being at an endpoint of the range as well as at any intervening value to the tenth of the unit of the lower limit of the range, as well as any subrange or sets of sub ranges of the range unless the context clearly dictates otherwise or any portion(s) of the stated range is specifically excluded. Where the stated range includes one or both endpoints of the range, ranges excluding either or both of those included endpoints are also included in the invention.

Certain numerical values described herein are preceded by “about”. In this context, “about” provides literal support for the exact numerical value that it precedes, the exact numerical value ±5%, as well as all other numerical values that are near to or approximately equal to that numerical value. Unless otherwise indicated a particular numerical value is included in “about” a specifically recited numerical value where the particular numerical value provides the substantial equivalent of the specifically recited numerical value in the context in which the specifically recited numerical value is presented. For example, a statement that something has the numerical value of “about 10” is to be interpreted as: the set of statements:
in some embodiments the numerical value is 10;
in some embodiments the numerical value is in the range of 9.5 to 10.5;
   and if from the context the person of ordinary skill in the art would understand that values within a certain range are substantially equivalent to 10 because the values with the range would be understood to provide substantially the same result as the value 10 then “about 10” also includes:
in some embodiments the numerical value is in the range of C to D where C and D are respectively lower and upper endpoints of the range that encompasses all of those values that provide a substantial equivalent to the value 10.

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

As will be apparent to those of skill in the art upon reading this disclosure, each of the individual embodiments described and illustrated herein has discrete components and features which may be readily separated from or combined with the features of any other described embodiment(s) without departing from the scope of the present invention.

Various features are described herein as being present in “some embodiments”. Such features are not mandatory and may not be present in all embodiments. Embodiments of the invention may include zero, any one or any combination of two or more of such features. All possible combinations of such features are contemplated by this disclosure even where such features are shown in different drawings and/or described in different sections or paragraphs. This is limited only to the extent that certain ones of such features are incompatible with other ones of such features in the sense that it would be impossible for a person of ordinary skill in the art to construct a practical embodiment that combines such incompatible features. Consequently, the description that “some embodiments” possess feature A and “some embodiments” possess feature B should be interpreted as an express indication that the inventors also contemplate embodiments which combine features A and B (unless the description states otherwise or features A and B are fundamentally incompatible). This is the case even if features A and B are illustrated in different drawings and/or mentioned in different paragraphs, sections or sentences.

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole.

What is claimed is:

1. A VANISH genetically engineered bacterium comprising an engineered nucleic acid comprising a non-native copy of an operon which encodes a betaine transport/import system, wherein the non-native copy of the operon is operatively linked to a promoter, and wherein the non-native copy of the operon is endogenous to the genome of a bacterial species from the Bacteroidaceae family.

2. The VANISH genetically engineered bacterium according to claim 1, wherein the non-native copy of the operon is endogenous to the bacterial species from the Bacteroides genus.

3. The VANISH genetically engineered bacterium according to claim 1, wherein the non-native copy of the operon comprises an opuA operon which comprises the genes opuAA, opuAB and opuAC which encode an ATPase, a transmembrane protein channel, and a periplasmic substrate-binding protein.

4. The VANISH genetically engineered bacterium according to claim 3, wherein the non-native copy of the operon comprises a nucleic acid sequence that has at least about 70% sequence identity to the nucleic acid sequence set forth in SEQ IQ NO: 1.

5. The VANISH genetically engineered bacterium according to claim 1, wherein the promoter comprises a nucleic acid sequence that is native to the genome of a bacterial species from the Bacteroides genus.

6. The VANISH genetically engineered bacterium according to claim 1, wherein the promoter comprises a nucleic acid sequence that is derived from a nucleic acid sequence that is native to the genome of a bacterial species from the Bacteroides genus.

7. The VANISH genetically engineered bacterium according to claim 1, wherein the nucleic acid sequence of the promoter comprises the -7/-10 consensus sequence of TAnnTTTG.

8. The VANISH genetically engineered bacterium according to claim 1, wherein the promoter comprises a nucleic acid sequence that has at least about 70% sequence identity to the nucleic acid sequence set forth in SEQ IQ NO: 2.

9. The VANISH genetically engineered bacterium according to claim 1, wherein the promoter comprises a nucleic acid sequence that has at least about 70% sequence identity to the nucleic acid sequence set forth in SEQ IQ NO: 35.

10. The VANISH genetically engineered bacterium according to claim 1, wherein the promoter is an inducible promoter.

11. The VANISH genetically engineered bacterium according to claim 1, wherein the promoter is a constitutive promoter.

12. The VANISH genetically engineered bacterium according to claim 1, wherein the engineered nucleic acid is integrated into a genome of a VANISH bacterium.

13. The VANISH genetically engineered bacterium according to claim 12, wherein the engineered nucleic acid is integrated into the genome of the VANISH bacterium at one or more recombinase recognition sites.

14. The VANISH genetically engineered bacterium according to claim 13, wherein the one or more recombinase recognition sites comprises an attBT site.

15. The VANISH genetically engineered bacterium according to claim 14, wherein the attBT site is located in the 3′ end of a serine tRNA (Ser-tRNA) gene.

16. The VANISH genetically engineered bacterium according to claim 1, wherein the VANISH bacterium contains an expression construct which comprises the promoter that is operatively linked to the non-native of the operon which encodes a betaine transport/import system.

17. The VANISH genetically engineered bacterium according to claim 16, wherein the expression construct comprises an integrative plasmid.

18. The VANISH genetically engineered bacterium according to claim 12, wherein the VANISH bacterium is from one of Muribaculaceae, Prevotellaceae, Spirochaetaceae and Succinivibrionaceae families.

19. Use of the VANISH genetically engineered bacterium according to claim 1 in treating, ameliorating and/or preventing disorders associated with high osmolality or malabsorption in a gut of a mammal.


SEQUENCE LISTING (40 sequences)

SEQ ID NO: 1 - DNA, 2942 bp, synthetic construct
  source 1..2942: mol_type = other DNA; note = opuA operon
atgagtaaaatagaaattaaagatctctatctggtttttggcaatgagaagcagaaggctttgaagatgttgaaagaaggtaaaacaaaaagtgaaatattgaaagcgaccggatgtacagttgcggtgaaagatgcgaatctttccattaatgaaggcgaaatattcgtcattatggggctgtccggtagtggaaagtccactttactccgctgcatcaaccgtttgatacgccctacttccggagaagtgattatcaatggaacagacatagccaaagtgtcggataaggaactgttgcagattcgccggaaagaactggctatggtctttcagaacttcggtctgcttcctcaccgttctgtcttgcataatatagctttcgggctcgaactacagggagtaaaaaagggagaacgggagcagaaagcgatggaaagtatgcaactggtggggctgaaaggctacgagaatcagatggtaagtgaactgtcgggtgggatgcagcagcgtgtcgggctggcgcgggctttggccaataatcccgaagtcctgttgatggatgaggccttctcggcactcgatcctttgatccgtgtgcagatgcaggatgaactgctgactttgcagtcgaagatgaaaaaaacgattgtctttattactcacgacctgagtgaagcgattaaattgggtgaccggattgctatcatgaaggatggagaaattgtacaaatcggtacatcggaagaaatcctgaccgaacctgccaacgcttatgtagaacgatttgtggagaatgtagatcgaagcaagattataacagcttcctctgttatggtagataaacccatagtggcacgtttgaagaaagaaggcccggaggtgctgatccgtaagatgcgcgaacggaatctgaccgttcttccggtagtcgattccaataatctgttagtaggagaggtgcggttgaatgacttgctgaaactacgaaaagagcaaatccgctccatcgagtccgtagtaaggcatgaagttcattcagtgctgggtgatacggtgctggaagatattcttccgttgatgacaaaaacgaactcgccaatctgggtcgtaaatgagaatcgggagtttgaaggcgtagtgccactttcctcattgatcatcgaagtaacgggaaaagataaagaagaaattaatgaaatcattcaaaacgcaatagaattatgataaatatcggacaatatatagagactgccattaattggctcacagaacattttgcttccttttttgatgctctcagcatggggatcggaggattcatagacggttttcagcacgtactgtttggaatacctttttatatcacaattgcagtgctggctgcgctcgcctggtttaagtcgggtaaggggacagccgtatttaccctcttgggtttattgcttatttacggcatgggcttttgggaagaaacgatgcagacattggcattggtactttcttccacttgtctggcattgcttttaggcgttccgttgggaatctggactgccaacagtgatcgttgtaacaagataatgcgccctgtcctcgactttatgcagacaatgcctgcctttgtctatctgattcctgccgttctattcttcggattgggaactgtgccgggagcttttgccaccatcatctttgcaatgcctcctgtggttcgtctgaccggtctgggcattcgtcaggttcctaaaaacgtagtagaagcctcccgctcttttggagctactccctggcagcttctttataaggtgcaattgcctttggcgttgcctactattctgacgggaatcaaccagacgatcatgatgtcactttcgatggtagtgattgctgccatgatttctgccggaggattgggagaaatcgtgctgaaaggtatcacccaaatgaaaatcggactcggatttgaaggcggtatcgcagtcgtgatattggcgatcgtgctggaccgcatcactcagggtatggcagataacagaaaaagtaaaaagaaataagaatcacaccaaagaaaggaataatcaatgaataagataaaaatgattgcggcaatgtttattgccggcatactcttaaccttcgtttcctgcggagacaacggaaagagtaagaagataagtatcgcttatgccaactggtcggaaggtattgcaatgacaaaccttgccaaagcgatttttgaagatcaggggtacgatgtaaagttgctgaatgccgaccttgcacctatatttacctctatctcccgtaagaaagcagacgtatttatggatgtctggatgcctgtcactatggaagattatatgaaacaatacggggataaactcgaagttatcggggatatttatgatggtgcccgtatcgggctggtggttccggattacgttacgatcaactcaatcgaagaactgaacgcggaaaaggaacgtttctccggtcagattgtcggtattgatgccggagccggtattatgaaagctaccgatcaggccatcaaagactatggattggattataaactgatgacttcgagcggacctgccatgactgcttccctaaagaaagcgattgataagaaagactggattgtcgtgacgggatggacgcctcactggatgttcgaccgtttcaagctgaaagttcttcaggacccgaagctgatatatggaaatacggaatccattcataccatagcatggaaaggttttagcgagaaagacccttttgctgccgaacttttgggcaacattcgtctgacagatgcggaaatcagctccttaatgactgctctggaagaggcgcagacaacggaaagacaggctgcccgtcaatggatggaagaacatcaggaactggtgaacagttggattcctgagaaagattga

SEQ ID NO: 2 - DNA, 79 bp, synthetic construct
  source 1..79: mol_type = other DNA; note = BT1311 (rpoD) derived promoter
gttttattagttgttaatatagtgttaaagttgcctaaatatgtatgttaacaaattatttgtcgtaactttgcactcc

SEQ ID NO: 3 - DNA, 82 bp, synthetic construct
  source 1..82: mol_type = other DNA; note = RBS1-LP
gatcacacaacatttaaaaaataacattatgaaagcactcgaaaagagattcaaaggtaatattaacaataatttattttca

SEQ ID NO: 4 - DNA, 22 bp, synthetic construct
  source 1..22: mol_type = other DNA; note = EM351 Assembly PCR primer for PBT1311UP (Outer F)
ctagcgatcgaggtctcaagtc

SEQ ID NO: 5 - DNA, 27 bp, synthetic construct
  source 1..27: mol_type = other DNA; note = EM383 Assembly PCR primer for PBT1311UP (Outer R)
atcgtaggtctccacgggttttattag

SEQ ID NO: 6 - DNA, 79 bp, synthetic construct
  source 1..79: mol_type = other DNA; note = EM388 Assembly PCR primer for PBT1311UP (Inner F)
gtaggtctccacgggttttattagttgttaatatagtgttaaagttgcctaaatatgtatgttaacaaattatttgtcg

SEQ ID NO: 7 - DNA, 71 bp, synthetic construct
  source 1..71: mol_type = other DNA; note = EM384 Assembly PCR primer for PBT1311UP (Inner R)
ctagcgatcgaggtctcaagtcggagtgcaaagttacgacaaataatttgttaacatacatatttaggcaa

SEQ ID NO: 8 - DNA, 35 bp, synthetic construct
  source 1..35: mol_type = other DNA; note = pEM189_F pNBU2 backbone F
gaaagattgaatcctagctgattagctcgagaagg

SEQ ID NO: 9 - DNA, 32 bp, synthetic construct
  source 1..32: mol_type = other DNA; note = pEM189_R pNBU2 backbone R
caactaataaaacgaattccaattcgaggggg

SEQ ID NO: 10 - DNA, 57 bp, synthetic construct
  source 1..57: mol_type = other DNA; note = opuA_F opuA operon F (Bt genome)
caataatttattttcaatgagtaaaatagaaattaaagatctctatctggtttttgg

SEQ ID NO: 11 - DNA, 41 bp, synthetic construct
  source 1..41: mol_type = other DNA; note = opuA_R opuA operon R (Bt genome)
tcagctaggattcaatctttctcaggaatccaactgttcac

SEQ ID NO: 12 - DNA, 60 bp, synthetic construct
  source 1..60: mol_type = other DNA; note = pBT1311_F pBT1311UP F
gaattggaattcgttttattagttgttaatatagtgttaaagttgcctaaatatgtatgt

SEQ ID NO: 13 - DNA, 60 bp, synthetic construct
  source 1..60: mol_type = other DNA; note = pBT1311_R pBT1311UP R
ctattttactcattgaaaataaattattgttaatattacctttgaatctcttttcgagtg

SEQ ID NO: 14 - DNA, 32 bp, synthetic construct
  source 1..32: mol_type = other DNA; note = JB131 no_prom_gibson_F
ggattcctgagaaagattgaatcctagctgat

SEQ ID NO: 15 - DNA, 46 bp, synthetic construct
  source 1..46: mol_type = other DNA; note = JB132 no_prom_gibson_R
ttaatttctattttactcatgaattccaattcgagggggatcaatt

SEQ ID NO: 16 - DNA, 61 bp, synthetic construct
  source 1..61: mol_type = other DNA; note = JB133 opuA_gibson_F
tccccctcgaattggaattcatgagtaaaatagaaattaaagatctctatctggtttttgg

SEQ ID NO: 17 - DNA, 32 bp, synthetic construct
  source 1..32: mol_type = other DNA; note = JB134 opuA_gibson_R
tcgagctaatcagctaggattcaatctttctc

SEQ ID NO: 18 - DNA, 22 bp, synthetic construct
  source 1..22: mol_type = other DNA; note = EM376 Bt genome at attBT1 site
cggagtgatgttatgacttacg

SEQ ID NO: 19 - DNA, 21 bp, synthetic construct
  source 1..21: mol_type = other DNA; note = EM377 Bt genome at attBT1/2 site
ccgactttacattcttgggtg

SEQ ID NO: 20 - DNA, 27 bp, synthetic construct
  source 1..27: mol_type = other DNA; note = EM378 Bt genome at attBT1 site
catgtctttatacttattataggctgc

SEQ ID NO: 21 - DNA, 24 bp, synthetic construct
  source 1..24: mol_type = other DNA; note = EM381 Bt genome at attBT2 site
ggaatggatatttttatgatgcgg

SEQ ID NO: 22 - DNA, 22 bp, synthetic construct
  source 1..22: mol_type = other DNA; note = EM382 Bt genome at attBT2 site
cctctatttccagttcacgttg

SEQ ID NO: 23 - DNA, 21 bp, synthetic construct
  source 1..21: mol_type = other DNA; note = EM377 MiNM06 genome at attBT1 site
ccgactttacattcttgggtg

SEQ ID NO: 24 - DNA, 29 bp, synthetic construct
  source 1..29: mol_type = other DNA; note = EM438 MiNM06 genome at attBT1 site
ctattaacatttagcctctacacatcatg

SEQ ID NO: 25 - DNA, 20 bp, synthetic construct
  source 1..20: mol_type = other DNA; note = EM439 MiNM06 genome at attBT1 site
accgggttttaaaagaaggg

SEQ ID NO: 26 - DNA, 21 bp, synthetic construct
  source 1..21: mol_type = other DNA; note = EM377 MiNM06 genome at attBT2 site
ccgactttacattcttgggtg

SEQ ID NO: 27 - DNA, 21 bp, synthetic construct
  source 1..21: mol_type = other DNA; note = EM440 MiNM06 genome at attBT2 site
ggttcgaatccctctctctcc

SEQ ID NO: 28 - DNA, 21 bp, synthetic construct
  source 1..21: mol_type = other DNA; note = EM441 MiNM06 genome at attBT2 site
gctcgcagagatatccatcag

SEQ ID NO: 29 - DNA, 6324 bp, synthetic construct
  source 1..6324: mol_type = other DNA; note = pEM189
cgggattaaaagtcggggattggtgaacaaaaaggtgtttctctctttaagagaaatatcgttttgctaaacagttgatattgaggtatcattttatcgtaaaagacatttttgctcaacaattgcttgacggaaatcaacaaattttagcattttgtaaaaaagtcgctatataatttggtgaattggagttattttcatatttttgcatcccgaagagtttctcttaaagagagaaacatcttttgcataccttttccgaccgaatttttatgtcgtaaagaggggctttgcagggggtggactcagaaagatgagaatagatgactattgtagttgaaacacatagaaagttgctgatatacagaccgatacgcatatcgggatgaaccatgagtacgttcttttctcaaaaaacataaatattcgaaaagagatgcaataaattaaggagaggttataatgaacaaagtaaatataaaagatagtcaaaattttattacttcaaaatatcacatagaaaaaataatgaattgcataagtttagatgaaaaagataacatctttgaaataggtgcagggaaaggtcattttactgctggattggtaaagagatgtaattttgtaacggcgatagaaattgattctaaattatgtgaggtaactcgtaataagctcttaaattatcctaactatcaaatagtaaatgatgatatactgaaatttacatttcctagccacaatccatataaaatatttggcagcataccttacaacataagcacaaatataattcgaaaaattgtttttgaaagttcagccacaataagttatttaatagtggaatatggttttgctaaaatgttattagatacaaacagatcactagcattgctgttaatggcagaggtagatatttctatattagcaaaaattcctaggtattatttccatccaaaacctaaagtggatagcacattaattgtattaaaaagaaagccagcaaaaatggcatttaaagagagaaaaaaatatgaaacttttgtaatgaaatgggttaacaaagagtacgaaaaactgtttacaaaaaatcaatttaataaagctttaaaacatgcgagaatatatgatataaacaatattagtttcgaacaatttgtatcgctatttaatagttataaaatatttaacggctaaaaacaataggccacatgcaactgtaaatgtttacgcgggtaccgacaccgcggtggaggggaattcccatgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatcctctacgccggacgcatcgtggccggatcaattccgttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcacggaattgatccccctcgaattggaattcgttcctcgattcagatctaaaggatccaatctagataaaacgaaaggctcagtcgaaagactgggcctttcgttttacaattgggctaccttttttttgttttgtttgcaatggttaatctattgttaaaatttaaagtttcacttgaactttcaaataatgttcttatatttccagtgtcgaaagaaacaaagtaggactgatcacacaacatttaaaaaataacattatgaaagcactcgaaaagagattcaaaggtaatattaacaataatttattttcagatcggatctccaggcatataaaacgaaaggctcagtcgaaagactgggcctttcgttttatctgttgtttgtcggtgaacgctctcctgagtaggacaaatcctaaagcactgctcgctttgttaaacgggttttattagttgttaatatagtgttaaagttgcctaaatatgtatgttaacaaattatttgtcgtaactttgcactccgactgatcacacaacatttaaaaaataacattatgaaagcactcgaaaagagattcaaaggtaatattaacaataatttattttcaatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggttccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaaggatctgattacaaggacgatgatgataaggactacaaagatgatgatgacaaagattataaggacgatgatgataaaggtcatcatcatcatcatcactaaatcctagctgattagctcgagaaggccatcctgacggatggccttttttttgtatcgagacgactagtgtaattgcctatcttccagtgatggaacagcatttgtgcattggctgcaacaatcagccttacttgtgcctgttctatttccgaaccgaccgcttgtatgaatccatcaaaattcgttttctctatgttggattccttgttgctcatattgtgatgataatttctacaaatatagtcattggtaactatctatgaaactgtttgatacttttatagttgattaaacttgttcatggcatttgccttaatatcatccgctatgtcaatgtagggtttcatagctttgtagtcgctgtgtcccgtccatttcatgaccacctgtgccgggattccgagagccagcgcattgcagatgaatgtccttcttcctgcatgggtactgagcaaagcgtatttgggtgtgacttcatcaatacgttcatttcccttgtagtaggtttcccgtacaggctcgttgatttctgccagttcgcccagctctttcaggtaatcgttcatcttctggttgctgatgacgggcagagccatgtaattctcgaaatggatgtccttgtatttgtccagtatggctttgctgtatttgttcagttcaatcgtcaggctgtcggcagtcttgactgtggttatttcgatgtggtcggacttcacatcgcttcttttcagattgcgaacatccgaataccgcaaactcgtaaagcagcagaacaggaaaacatcacgcacacgttccaggtattgcttatccttgggtatctggtagtctttcagcttgttcagttcatcccaagtcaggaagattacttttttcgaggtggttttcagtttcggtttgaacgtatcgtatgcaatgttctgatgatgtcctttcttgaagctccagcgcaggaaccatttgaggaatcccatttgcttgccgatggtgctgtttctcatatccttggtgtcacgcaggaagttgacgtattcgttcaatccaaactcgttgaaatagttgaacgttgcatcctccttgaactctttgaggtggttcctcactgctgcaaatttttcataggtggatgccgtccagttattctggttaccgcactcttttacaaactcatcgaacacctcccaaaagctgacaggggcttcttccggctgttcttcgctggtgtctttcattctcatgttgaaagcttccttcaactgttgggtcgttggcatgacctcctgcacctcaaattccttgaaaatattctggatttcggcatagtatttcagcaagtccgtattgatttcggctgcactttgctttagcttgttggtacatccgctctttacccgctgcttatctgcatcccatttggctacgtcaatccggtagcccgttgtaaactcgatgcgttggctggcaaagatgacacgcatacggatgggtacgttctctacgattggcacaccgttctttttccggctctccaatgcaaaaatgatgttgcgcttgatattcataattgggtgcgtttgaaattctacacccaaatatacacccaattattgagatagcaaaagacatttagaaacatttacttttactctatattgtaatttacacttgattatcagtcgtttgcagtcttatgatattctgtgaaagtataagttcgagagcctgtctctccgcaaaaaacgctgaaaatcagcagattgcaaaacaaacaccctgttttacacccaagaatgtaaagtcggctgtttttgttttatttaagataatacaaccactacataataaaagagtagcgatattaaaagaatccgatgagaaaagactaatatttatctatccattcagtttgatttttcaggactttacatcgtcctgaaagtatttgttggtaccgaggacgcgacagctgcagacgcgttatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatggccg

SEQ ID NO: 30 - DNA, 8036 bp, synthetic construct
  source 1..8036: mol_type = other DNA; note = pJB0A
cgggattaaaagtcggggattggtgaacaaaaaggtgtttctctctttaagagaaatatcgttttgctaaacagttgatattgaggtatcattttatcgtaaaagacatttttgctcaacaattgcttgacggaaatcaacaaattttagcattttgtaaaaaagtcgctatataatttggtgaattggagttattttcatatttttgcatcccgaagagtttctcttaaagagagaaacatcttttgcataccttttccgaccgaatttttatgtcgtaaagaggggctttgcagggggtggactcagaaagatgagaatagatgactattgtagttgaaacacatagaaagttgctgatatacagaccgatacgcatatcgggatgaaccatgagtacgttcttttctcaaaaaacataaatattcgaaaagagatgcaataaattaaggagaggttataatgaacaaagtaaatataaaagatagtcaaaattttattacttcaaaatatcacatagaaaaaataatgaattgcataagtttagatgaaaaagataacatctttgaaataggtgcagggaaaggtcattttactgctggattggtaaagagatgtaattttgtaacggcgatagaaattgattctaaattatgtgaggtaactcgtaataagctcttaaattatcctaactatcaaatagtaaatgatgatatactgaaatttacatttcctagccacaatccatataaaatatttggcagcataccttacaacataagcacaaatataattcgaaaaattgtttttgaaagttcagccacaataagttatttaatagtggaatatggttttgctaaaatgttattagatacaaacagatcactagcattgctgttaatggcagaggtagatatttctatattagcaaaaattcctaggtattatttccatccaaaacctaaagtggatagcacattaattgtattaaaaagaaagccagcaaaaatggcatttaaagagagaaaaaaatatgaaacttttgtaatgaaatgggttaacaaagagtacgaaaaactgtttacaaaaaatcaatttaataaagctttaaaacatgcgagaatatatgatataaacaatattagtttcgaacaatttgtatcgctatttaatagttataaaatatttaacggctaaaaacaataggccacatgcaactgtaaatgtttacgcgggtaccgacaccgcggtggaggggaattcccatgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatcctctacgccggacgcatcgtggccggatcaattccgttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcacggaattgatccccctcgaattggaattcgttttattagttgttaatatagtgttaaagttgcctaaatatgtatgttaacaaattatttgtcgtaactttgcactccgactgatcacacaacatttaaaaaataacattatgaaagcactcgaaaagagattcaaaggtaatattaacaataatttattttcaatgagtaaaatagaaattaaagatctctatctggtttttggcaatgagaagcagaaggctttgaagatgttgaaagaaggtaaaacaaaaagtgaaatattgaaagcgaccggatgtacagttgcggtgaaagatgcgaatctttccattaatgaaggcgaaatattcgtcattatggggctgtccggtagtggaaagtccactttactccgctgcatcaaccgtttgatacgccctacttccggagaagtgattatcaatggaacagacatagccaaagtgtcggataaggaactgttgcagattcgccggaaagaactggctatggtctttcagaacttcggtctgcttcctcaccgttctgtcttgcataatatagctttcgggctcgaactacagggagtaaaaaagggagaacgggagcagaaagcgatggaaagtatgcaactggtggggctgaaaggctacgagaatcagatggtaagtgaactgtcgggtgggatgcagcagcgtgtcgggctggcgcgggctttggccaataatcccgaagtcctgttgatggatgaggccttctcggcactcgatcctttgatccgtgtgcagatgcaggatgaactgctgactttgcagtcgaagatgaaaaaaacgattgtctttattactcacgacctgagtgaagcgattaaattgggtgaccggattgctatcatgaaggatggagaaattgtacaaatcggtacatcggaagaaatcctgaccgaacctgccaacgcttatgtagaacgatttgtggagaatgtagatcgaagcaagattataacagcttcctctgttatggtagataaacccatagtggcacgtttgaagaaagaaggcccggaggtgctgatccgtaagatgcgcgaacggaatctgaccgttcttccggtagtcgattccaataatctgttagtaggagaggtgcggttgaatgacttgctgaaactacgaaaagagcaaatccgctccatcgagtccgtagtaaggcatgaagttcattcagtgctgggtgatacggtgctggaagatattcttccgttgatgacaaaaacgaactcgccaatctgggtcgtaaatgagaatcgggagtttgaaggcgtagtgccactttcctcattgatcatcgaagtaacgggaaaagataaagaagaaattaatgaaatcattcaaaacgcaatagaattatgataaatatcggacaatatatagagactgccattaattggctcacagaacattttgcttccttttttgatgctctcagcatggggatcggaggattcatagacggttttcagcacgtactgtttggaatacctttttatatcacaattgcagtgctggctgcgctcgcctggtttaagtcgggtaaggggacagccgtatttaccctcttgggtttattgcttatttacggcatgggcttttgggaagaaacgatgcagacattggcattggtactttcttccacttgtctggcattgcttttaggcgttccgttgggaatctggactgccaacagtgatcgttgtaacaagataatgcgccctgtcctcgactttatgcagacaatgcctgcctttgtctatctgattcctgccgttctattcttcggattgggaactgtgccgggagcttttgccaccatcatctttgcaatgcctcctgtggttcgtctgaccggtctgggcattcgtcaggttcctaaaaacgtagtagaagcctcccgctcttttggagctactccctggcagcttctttataaggtgcaattgcctttggcgttgcctactattctgacgggaatcaaccagacgatcatgatgtcactttcgatggtagtgattgctgccatgatttctgccggaggattgggagaaatcgtgctgaaaggtatcacccaaatgaaaatcggactcggatttgaaggcggtatcgcagtcgtgatattggcgatcgtgctggaccgcatcactcagggtatggcagataacagaaaaagtaaaaagaaataagaatcacaccaaagaaaggaataatcaatgaataagataaaaatgattgcggcaatgtttattgccggcatactcttaaccttcgtttcctgcggagacaacggaaagagtaagaagataagtatcgcttatgccaactggtcggaaggtattgcaatgacaaaccttgccaaagcgatttttgaagatcaggggtacgatgtaaagttgctgaatgccgaccttgcacctatatttacctctatctcccgtaagaaagcagacgtatttatggatgtctggatgcctgtcactatggaagattatatgaaacaatacggggataaactcgaagttatcggggatatttatgatggtgcccgtatcgggctggtggttccggattacgttacgatcaactcaatcgaagaactgaacgcggaaaaggaacgtttctccggtcagattgtcggtattgatgccggagccggtattatgaaagctaccgatcaggccatcaaagactatggattggattataaactgatgacttcgagcggacctgccatgactgcttccctaaagaaagcgattgataagaaagactggattgtcgtgacgggatggacgcctcactggatgttcgaccgtttcaagctgaaagttcttcaggacccgaagctgatatatggaaatacggaatccattcataccatagcatggaaaggttttagcgagaaagacccttttgctgccgaacttttgggcaacattcgtctgacagatgcggaaatcagctccttaatgactgctctggaagaggcgcagacaacggaaagacaggctgcccgtcaatggatggaagaacatcaggaactggtgaacagttggattcctgagaaagattgaatcctagctgattagctcgagaaggccatcctgacggatggccttttttttgtatcgagacgactagtgtaattgcctatcttccagtgatggaacagcatttgtgcattggctgcaacaatcagccttacttgtgcctgttctatttccgaaccgaccgcttgtatgaatccatcaaaattcgttttctctatgttggattccttgttgctcatattgtgatgataatttctacaaatatagtcattggtaactatctatgaaactgtttgatacttttatagttgattaaacttgttcatggcatttgccttaatatcatccgctatgtcaatgtagggtttcatagctttgtagtcgctgtgtcccgtccatttcatgaccacctgtgccgggattccgagagccagcgcattgcagatgaatgtccttcttcctgcatgggtactgagcaaagcgtatttgggtgtgacttcatcaatacgttcatttcccttgtagtaggtttcccgtacaggctcgttgatttctgccagttcgcccagctctttcaggtaatcgttcatcttctggttgctgatgacgggcagagccatgtaattctcgaaatggatgtccttgtatttgtccagtatggctttgctgtatttgttcagttcaatcgtcaggctgtcggcagtcttgactgtggttatttcgatgtggtcggacttcacatcgcttcttttcagattgcgaacatccgaataccgcaaactcgtaaagcagcagaacaggaaaacatcacgcacacgttccaggtattgcttatccttgggtatctggtagtctttcagcttgttcagttcatcccaagtcaggaagattacttttttcgaggtggttttcagtttcggtttgaacgtatcgtatgcaatgttctgatgatgtcctttcttgaagctccagcgcaggaaccatttgaggaatcccatttgcttgccgatggtgctgtttctcatatccttggtgtcacgcaggaagttgacgtattcgttcaatccaaactcgttgaaatagttgaacgttgcatcctccttgaactctttgaggtggttcctcactgctgcaaatttttcataggtggatgccgtccagttattctggttaccgcactcttttacaaactcatcgaacacctcccaaaagctgacaggggcttcttccggctgttcttcgctggtgtctttcattctcatgttgaaagcttccttcaactgttgggtcgttggcatgacctcctgcacctcaaattccttgaaaatattctggatttcggcatagtatttcagcaagtccgtattgatttcggctgcactttgctttagcttgttggtacatccgctctttacccgctgcttatctgcatcccatttggctacgtcaatccggtagcccgttgtaaactcgatgcgttggctggcaaagatgacacgcatacggatgggtacgttctctacgattggcacaccgttctttttccggctctccaatgcaaaaatgatgttgcgcttgatattcataattgggtgcgtttgaaattctacacccaaatatacacccaattattgagatagcaaaagacatttagaaacatttacttttactctatattgtaatttacacttgattatcagtcgtttgcagtcttatgatattctgtgaaagtataagttcgagagcctgtctctccgcaaaaaacgctgaaaatcagcagattgcaaaacaaacaccctgttttacacccaagaatgtaaagtcggctgtttttgttttatttaagataatacaaccactacataataaaagagtagcgatattaaaagaatccgatgagaaaagactaatatttatctatccattcagtttgatttttcaggactttacatcgtcctgaaagtatttgttggtaccgaggacgcgacagctgcagacgcgttatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatggccg

SEQ ID NO: 31 - DNA, 7871 bp, synthetic construct
  source 1..7871: mol_type = other DNA; note = pJB061
cgggattaaaagtcggggattggtgaacaaaaaggtgtttctctctttaagagaaatatcgttttgctaaacagttgatattgaggtatcattttatcgtaaaagacatttttgctcaacaattgcttgacggaaatcaacaaattttagcattttgtaaaaaagtcgctatataatttggtgaattggagttattttcatatttttgcatcccgaagagtttctcttaaagagagaaacatcttttgcataccttttccgaccgaatttttatgtcgtaaagaggggctttgcagggggtggactcagaaagatgagaatagatgactattgtagttgaaacacatagaaagttgctgatatacagaccgatacgcatatcgggatgaaccatgagtacgttcttttctcaaaaaacataaatattcgaaaagagatgcaataaattaaggagaggttataatgaacaaagtaaatataaaagatagtcaaaattttattacttcaaaatatcacatagaaaaaataatgaattgcataagtttagatgaaaaagataacatctttgaaataggtgcagggaaaggtcattttactgctggattggtaaagagatgtaattttgtaacggcgatagaaattgattctaaattatgtgaggtaactcgtaataagctcttaaattatcctaactatcaaatagtaaatgatgatatactgaaatttacatttcctagccacaatccatataaaatatttggcagcataccttacaacataagcacaaatataattcgaaaaattgtttttgaaagttcagccacaataagttatttaatagtggaatatggttttgctaaaatgttattagatacaaacagatcactagcattgctgttaatggcagaggtagatatttctatattagcaaaaattcctaggtattatttccatccaaaacctaaagtggatagcacattaattgtattaaaaagaaagccagcaaaaatggcatttaaagagagaaaaaaatatgaaacttttgtaatgaaatgggttaacaaagagtacgaaaaactgtttacaaaaaatcaatttaataaagctttaaaacatgcgagaatatatgatataaacaatattagtttcgaacaatttgtatcgctatttaatagttataaaatatttaacggctaaaaacaataggccacatgcaactgtaaatgtttacgcgggtaccgacaccgcggtggaggggaattcccatgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatcctctacgccggacgcatcgtggccggatcaattccgttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcacggaattgatccccctcgaattggaattcatgagtaaaatagaaattaaagatctctatctggtttttggcaatgagaagcagaaggctttgaagatgttgaaagaaggtaaaacaaaaagtgaaatattgaaagcgaccggatgtacagttgcggtgaaagatgcgaatctttccattaatgaaggcgaaatattcgtcattatggggctgtccggtagtggaaagtccactttactccgctgcatcaaccgtttgatacgccctacttccggagaagtgattatcaatggaacagacatagccaaagtgtcggataaggaactgttgcagattcgccggaaagaactggctatggtctttcagaacttcggtctgcttcctcaccgttctgtcttgcataatatagctttcgggctcgaactacagggagtaaaaaagggagaacgggagcagaaagcgatggaaagtatgcaactggtggggctgaaaggctacgagaatcagatggtaagtgaactgtcgggtgggatgcagcagcgtgtcgggctggcgcgggctttggccaataatcccgaagtcctgttgatggatgaggccttctcggcactcgatcctttgatccgtgtgcagatgcaggatgaactgctgactttgcagtcgaagatgaaaaaaacgattgtctttattactcacgacctgagtgaagcgattaaattgggtgaccggattgctatcatgaaggatggagaaattgtacaaatcggtacatcggaagaaatcctgaccgaacctgccaacgcttatgtagaacgatttgtggagaatgtagatcgaagcaagattataacagcttcctctgttatggtagataaacccatagtggcacgtttgaagaaagaaggcccggaggtgctgatccgtaagatgcgcgaacggaatctgaccgttcttccggtagtcgattccaataatctgttagtaggagaggtgcggttgaatgacttgctgaaactacgaaaagagcaaatccgctccatcgagtccgtagtaaggcatgaagttcattcagtgctgggtgatacggtgctggaagatattcttccgttgatgacaaaaacgaactcgccaatctgggtcgtaaatgagaatcgggagtttgaaggcgtagtgccactttcctcattgatcatcgaagtaacgggaaaagataaagaagaaattaatgaaatcattcaaaacgcaatagaattatgataaatatcggacaatatatagagactgccattaattggctcacagaacattttgcttccttttttgatgctctcagcatggggatcggaggattcatagacggttttcagcacgtactgtttggaatacctttttatatcacaattgcagtgctggctgcgctcgcctggtttaagtcgggtaaggggacagccgtatttaccctcttgggtttattgcttatttacggcatgggcttttgggaagaaacgatgcagacattggcattggtactttcttccacttgtctggcattgcttttaggcgttccgttgggaatctggactgccaacagtgatcgttgtaacaagataatgcgccctgtcctcgactttatgcagacaatgcctgcctttgtctatctgattcctgccgttctattcttcggattgggaactgtgccgggagcttttgccaccatcatctttgcaatgcctcctgtggttcgtctgaccggtctgggcattcgtcaggttcctaaaaacgtagtagaagcctcccgctcttttggagctactccctggcagcttctttataaggtgcaattgcctttggcgttgcctactattctgacgggaatcaaccagacgatcatgatgtcactttcgatggtagtgattgctgccatgatttctgccggaggattgggagaaatcgtgctgaaaggtatcacccaaatgaaaatcggactcggatttgaaggcggtatcgcagtcgtgatattggcgatcgtgctggaccgcatcactcagggtatggcagataacagaaaaagtaaaaagaaataagaatcacaccaaagaaaggaataatcaatgaataagataaaaatgattgcggcaatgtttattgccggcatactcttaaccttcgtttcctgcggagacaacggaaagagtaagaagataagtatcgcttatgccaactggtcggaaggtattgcaatgacaaaccttgccaaagcgatttttgaagatcaggggtacgatgtaaagttgctgaatgccgaccttgcacctatatttacctctatctcccgtaagaaagcagacgtatttatggatgtctggatgcctgtcactatggaagattatatgaaacaatacggggataaactcgaagttatcggggatatttatgatggtgcccgtatcgggctggtggttccggattacgttacgatcaactcaatcgaagaactgaacgcggaaaaggaacgtttctccggtcagattgtcggtattgatgccggagccggtattatgaaagctaccgatcaggccatcaaagactatggattggattataaactgatgacttcgagcggacctgccatgactgcttccctaaagaaagcgattgataagaaagactggattgtcgtgacgggatggacgcctcactggatgttcgaccgtttcaagctgaaagttcttcaggacccgaagctgatatatggaaatacggaatccattcataccatagcatggaaaggttttagcgagaaagacccttttgctgccgaacttttgggcaacattcgtctgacagatgcggaaatcagctccttaatgactgctctggaagaggcgcagacaacggaaagacaggctgcccgtcaatggatggaagaacatcaggaactggtgaacagttggattcctgagaaagattgaatcctagctgattagctcgagaaggccatcctgacggatggccttttttttgtatcgagacgactagtgtaattgcctatcttccagtgatggaacagcatttgtgcattggctgcaacaatcagccttacttgtgcctgttctatttccgaaccgaccgcttgtatgaatccatcaaaattcgttttctctatgttggattccttgttgctcatattgtgatgataatttctacaaatatagtcattggtaactatctatgaaactgtttgatacttttatagttgattaaacttgttcatggcatttgccttaatatcatccgctatgtcaatgtagggtttcatagctttgtagtcgctgtgtcccgtccatttcatgaccacctgtgccgggattccgagagccagcgcattgcagatgaatgtccttcttcctgcatgggtactgagcaaagcgtatttgggtgtgacttcatcaatacgttcatttcccttgtagtaggtttcccgtacaggctcgttgatttctgccagttcgcccagctctttcaggtaatcgttcatcttctggttgctgatgacgggcagagccatgtaattctcgaaatggatgtccttgtatttgtccagtatggctttgctgtatttgttcagttcaatcgtcaggctgtcggcagtcttgactgtggttatttcgatgtggtcggacttcacatcgcttcttttcagattgcgaacatccgaataccgcaaactcgtaaagcagcagaacaggaaaacatcacgcacacgttccaggtattgcttatccttgggtatctggtagtctttcagcttgttcagttcatcccaagtcaggaagattacttttttcgaggtggttttcagtttcggtttgaacgtatcgtatgcaatgttctgatgatgtcctttcttgaagctccagcgcaggaaccatttgaggaatcccatttgcttgccgatggtgctgtttctcatatccttggtgtcacgcaggaagttgacgtattcgttcaatccaaactcgttgaaatagttgaacgttgcatcctccttgaactctttgaggtggttcctcactgctgcaaatttttcataggtggatgccgtccagttattctggttaccgcactcttttacaaactcatcgaacacctcccaaaagctgacaggggcttcttccggctgttcttcgctggtgtctttcattctcatgttgaaagcttccttcaactgttgggtcgttggcatgacctcctgcacctcaaattccttgaaaatattctggatttcggcatagtatttcagcaagtccgtattgatttcggctgcactttgctttagcttgttggtacatccgctctttacccgctgcttatctgcatcccatttggctacgtcaatccggtagcccgttgtaaactcgatgcgttggctggcaaagatgacacgcatacggatgggtacgttctctacgattggcacaccgttctttttccggctctccaatgcaaaaatgatgttgcgcttgatattcataattgggtgcgtttgaaattctacacccaaatatacacccaattattgagatagcaaaagacatttagaaacatttacttttactctatattgtaatttacacttgattatcagtcgtttgcagtcttatgatattctgtgaaagtataagttcgagagcctgtctctccgcaaaaaacgctgaaaatcagcagattgcaaaacaaacaccctgttttacacccaagaatgtaaagtcggctgtttttgttttatttaagataatacaaccactacataataaaagagtagcgatattaaaagaatccgatgagaaaagactaatatttatctatccattcagtttgatttttcaggactttacatcgtcctgaaagtatttgttggtaccgaggacgcgacagctgcagacgcgttatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatggccg

SEQ ID NO: 32 - DNA, 5722 bp, synthetic construct
  source 1..5722: mol_type = other DNA; note = pDP01
agcggccgccaccgcggtggaggggaattcccatgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatcctctacgccggacgcatcgtggccggatcaattccgttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcacggaattgatccccctcgaattgacgcgttatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatggccgcgggattaaaagtcggggattggtgaacaaaaaggtgtttctctctttaagagaaatatcgttttgctaaacagttgatattgaggtatcattttatcgtaaaagacatttttgctcaacaattgcttgacggaaatcaacaaattttagcattttgtaaaaaagtcgctatataatttggtgaattggagttattttcatatttttgcatcccgaagagtttctcttaaagagagaaacatcttttgcataccttttccgaccgaatttttatgtcgtaaagaggggctttgcagggggtggactcagaaagatgagaatagatgactattgtagttgaaacacatagaaagttgctgatatacagaccgatacgcatatcgggatgaaccatgagtacgttcttttctcaaaaaacataaatattcgaaaagagatgcaataaattaaggagaggttataatgaacaaagtaaatataaaagatagtcaaaattttattacttcaaaatatcacatagaaaaaataatgaattgcataagtttagatgaaaaagataacatctttgaaataggtgcagggaaaggtcattttactgctggattggtaaagagatgtaattttgtaacggcgatagaaattgattctaaattatgtgaggtaactcgtaataagctcttaaattatcctaactatcaaatagtaaatgatgatatactgaaatttacatttcctagccacaatccatataaaatatttggcagcataccttacaacataagcacaaatataattcgaaaaattgtttttgaaagttcagccacaataagttatttaatagtggaatatggttttgctaaaatgttattagatacaaacagatcactagcattgctgttaatggcagaggtagatatttctatattagcaaaaattcctaggtattatttccatccaaaacctaaagtggatagcacattaattgtattaaaaagaaagccagcaaaaatggcatttaaagagagaaaaaaatatgaaacttttgtaatgaaatgggttaacaaagagtacgaaaaactgtttacaaaaaatcaatttaataaagctttaaaacatgcgagaatatatgatataaacaatattagtttcgaacaatttgtatcgctatttaatagttataaaatatttaacggctaaaaacaataggccacatgcaactgtaaatgtttacgcgtcctcggtaccgcttcttccacaacagtctgcggttcctgtactatcacaggttcatcttctcccggtgctgtcgcttcattctcttccacgaccttctcttctatcaccggacattcaacttcctctattacaggggcttgttcttcaacaatgggggcttcactttccgcttccgctacaggagaaggcgaggcttccaccggcacagcacttatctcttccgacaactgttccaaacgctcctgcatacgtaggatgctccgcttcaacagttcagacaaagtctgagtcggctctttagaaaacgtattcatgagtagcttcagctcatgaacatccagctcaatatctgttaatagcttctgtttcatttccatccattgttattatggtgcgaatgtaggaataatttatgcaataacttaaaataatggataatttaacagttcttgtacatcgaattgtttacctttgtccactcatttatttaatcaagaaagcacaaaaatcacatggtattattttcagaagaccacatacaagagaccagaagaagaggtagaatagaggtaatctgcggttcaatgttctccggaaagacggaggaattgattcgtaggatgaagagagccaagttcgcacgccagcgtgtagagatattcaaaccggccatcgacacccgttattcggaagaagacgtggtatcacacgacagtcattccatcgcgtccacccctatcgattcatcggctagtatcctgttgttcacttctgaaatagatgtagtcggcatcgacgaagcacagttctttgatgacggactgattgacgtatgcaggcagcttgccaataacggcatccgtgtcatcatcgccggactggatatggactttaaagggaatcccttcggaccgatgcctcaattatgtgccattgccgatgaagtttccaaagttcatgcgatctgcgtgaagtgcggtgatctggcatctttctctcatcgtaccgtcaagaatgacaagcaggtacttctaggagagacagccgaatacgagccgctttgcagggagtgctacctccgtgcacggggagaggacggacagaagatataaactctatttaaaacatatatggaaagaaagaagataacattcgagtcgacggtatcgataagcttgatatcgaattcctgcagcccggggggtccagcacgatcgccaatatcacgactgcgataccgccttcaaatccgagtccgattttcatttgggtgatacctttcagcacgatttctcccaatcctccggcagaaatcatggcagcaatcactaccatcgaaagtgacatcatgatcgtctggttgattcccgtcagaatagtaggcaacgccaaaggcaattgcaccttataaagaagctgccagggagtagctccaaaagagcgggaggcttctactacgtttttaggaacctgacgaatgcccagaccggtcagacgaaccacaggaggcattgcaaagatgatggtggcaaaagctcccggcacagttcccaatccgaagaatagaacggcaggaatcagatagacaaaggcaggcattgtctgcataaagtcgaggacagggcgcattatcttgttacaacgatcactgttggcagtccagattcccaacggaacgcctaaaagcaatgccagacaagtggaagaaagtaccaatgccaatgtctgcatcgtttcttcccaaaagcccatgccgtaaataagcaataaacccaagagggtaaatacggctgtccccttacccgacttaaaccaggcgagcgcagccagcactgcaattgtgatataaaaaggtattccaaacagtacgtgctgaaaaccgtctatgaatcctccgatccccatgctgagagcatcaaaaaaggaagcaaaatgttctgtgagccaattaatggcagtctctatatattgtccgatatttatcacatacaataaaatggtttaggttattaaaataatatacacagcaagacaagaacaaacgataacaggaatctttctttatatcgtataaagttctgatttaaataagggacaaataacaatgaatcccgaaataattaataaaatgattattgacagactgagtaattaaaaaaggaatagaatttgataagataatcctttcaacgaatagcaaaaaatagcatttttatgtataaaaacagcctgttccagtatcttgctggcacaaagatacaaaaaacggattaaacatacaaattatattcaaaatccggggcatacagagggctttatttgtaatgatatggaaagtttccgggtaatattcccctatcactcagcaaaatagaacaaaccggcagataaatataaaataagtttgaatatttcacttaaacaccgtatatttggcgtcaataactaaactagatttatatgttcaacagggctattacaattttcctggggaaggataattgtcattacacaacaaaagaaaaacaaacaggtactgccacactagacgacaaaagccaagtacagttccttagtaaaaagaaccccaaaatgccgtttttctatagccggcggaatctctacatgataggtttcctgctcgcttttatagtgactctgctcgaatttatacgaggacggcatcaaaactttatggtcttctcggacgctactcaattcttctggcaaggcatcagcccatacacaactgaatttgtagaagctca

SEQ ID NO: 33 - DNA, 5722 bp, synthetic construct
  source 1..5722: mol_type = other DNA; note = pDP02
agcggccgccaccgcggtggaggggaattcccatgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatcctctacgccggacgcatcgtggccggatcaattccgttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcacggaattgatccccctcgaattgacgcgttatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatggccgcgggattaaaagtcggggattggtgaacaaaaaggtgtttctctctttaagagaaatatcgttttgctaaacagttgatattgaggtatcattttatcgtaaaagacatttttgctcaacaattgcttgacggaaatcaacaaattttagcattttgtaaaaaagtcgctatataatttggtgaattggagttattttcatatttttgcatcccgaagagtttctcttaaagagagaaacatcttttgcataccttttccgaccgaatttttatgtcgtaaagaggggctttgcagggggtggactcagaaagatgagaatagatgactattgtagttgaaacacatagaaagttgctgatatacagaccgatacgcatatcgggatgaaccatgagtacgttcttttctcaaaaaacataaatattcgaaaagagatgcaataaattaaggagaggttataatgaacaaagtaaatataaaagatagtcaaaattttattacttcaaaatatcacatagaaaaaataatgaattgcataagtttagatgaaaaagataacatctttgaaataggtgcagggaaaggtcattttactgctggattggtaaagagatgtaattttgtaacggcgatagaaattgattctaaattatgtgaggtaactcgtaataagctcttaaattatcctaactatcaaatagtaaatgatgatatactgaaatttacatttcctagccacaatccatataaaatatttggcagcataccttacaacataagcacaaatataattcgaaaaattgtttttgaaagttcagccacaataagttatttaatagtggaatatggttttgctaaaatgttattagatacaaacagatcactagcattgctgttaatggcagaggtagatatttctatattagcaaaaattcctaggtattatttccatccaaaacctaaagtggatagcacattaattgtattaaaaagaaagccagcaaaaatggcatttaaagagagaaaaaaatatgaaacttttgtaatgaaatgggttaacaaagagtacgaaaaactgtttacaaaaaatcaatttaataaagctttaaaacatgcgagaatatatgatataaacaatattagtttcgaacaatttgtatcgctatttaatagttataaaatatttaacggctaaaaacaataggccacatgcaactgtaaatgtttacgcgtcctcggtaccgcttcttccacaacagtctgcggttcctgtactatcacaggttcatcttctcccggtgctgtcgcttcattctcttccacgaccttctcttctatcaccggacattcaacttcctctattacaggggcttgttcttcaacaatgggggcttcactttccgcttccgctacaggagaaggcgaggcttccaccggcacagcacttatctcttccgacaactgttccaaacgctcctgcatacgtaggatgctccgcttcaacagttcagacaaagtctgagtcggctctttagaaaacgtattcatgagtagcttcagctcatgaacatccagctcaatatctgttaatagcttctgtttcatttccatccattgttattatggtgcgaatgtaggaataatttatgcaataacttaaaataatggataatttaacagttcttgtacatcgaattgtttacctttgtccactcatttatttaatcaagaaagcacaaaaatcacatggtattattttcagaagaccacatacaagagaccagaagaagaggtagaatagaggtaatctgcggttcaatgttctccggaaagacggaggaattgattcgtaggatgaagagagccaagttcgcacgccagcgtgtagagatattcaaaccggccatcgacacccgttattcggaagaagacgtggtatcacacgacagtcattccatcgcgtccacccctatcgattcatcggctagtatcctgttgttcacttctgaaatagatgtagtcggcatcgacgaagcacagttctttgatgacggactgattgacgtatgcaggcagcttgccaataacggcatccgtgtcatcatcgccggactggatatggactttaaagggaatcccttcggaccgatgcctcaattatgtgccattgccgatgaagtttccaaagttcatgcgatctgcgtgaagtgcggtgatctggcatctttctctcatcgtaccgtcaagaatgacaagcaggtacttctaggagagacagccgaatacgagccgctttgcagggagtgctacctccgtgcacggggagaggacggacagaagatataaactctatttaaaacatatatggaaagaaagaagataacattcgagtcgacggtatcgataagcttgatatcgaattcctgcagcccgggggatggctactacttacggttatgtatatgttgctcagattgctatgggtgctgaccaggctcagactctgaaagctatccgtgaagctgaagcatatccgggaccatcactgatcatcgcttacgctccatgtatcaaccacggtctgaaagccggtatgggtaagagccaggaagaagaagaaaaggctgttaagtgcggttactggcacttgtggcgttacaaccctgctttggaagaagaaggcaagaatccgttccaactggatagcaaagaacctaactgggaagacttccaaggtttcttgaagggtgaagttcgttacgcttctgtaatgaaacaatatcctgctgaagctgaagaactgtttaaagctgctgaagaaaacgctaaatggcgttacaacagctacaaacgtcttgccagagaaaactggggagctgatacagctgaataataagtagaaatactttgtacataataaaaggcaggaatcctggaaacagattcctgccttttttgtttatatcttagaagtaacgattacttctcacgaagatattcattaaaactctaaagatatgatcggatttgttatttggattatcggtttgatactgactttcagagctgccatagagatttggaatgtaaatgcggctgtagagaaaagacttatagcgattgtattaatcgttcttaccagctggctgggactacttttctactacttctatggtaaagaaagaatggcgggatggttgaagtagtgtttgcatatcacattgccgcaatcatttttatcttattcattgattattcctttctttggtgtgattcttatttctttttactttttctgttatctgccataccctgagtgatgcggtccagcacgatcgccaatatcacgactgcgataccgccttcaaatccgagtccgattttcatttgggtgatacctttcagcacgatttctcccaatcctccggcagaaatcatggcagcaatcactaccatcgaaagtgacatcatgatcgtctggttgattcccgtcagaatagtaggcaacgccaaaggcaattgcaccttataaagaagctgccagggagtagctccaaaagagcgggaggcttctactacgtttttaggaacctgacgaatgcccagaccggtcagacgaaccacaggaggcattgcaaagatgatggtggcaaaagctcccggcacagttcccaatccgaagaatagaacggcaggaatcagatagacaaaggcaggcattgtctgcataaagtcgaggacagggcgcattatcttgttacaacgatcactgttggcagtccagattcccaacggaacgcctaaaagcaatgccagacaagtggaagaaagtaccaatgccaatgtctgcatcgtttcttcccaaaagcccatgccgtaaataagcaataaacccaagagggtaaatacggctgtccccttacccgacttaaaccaggcgagcgcagccagcactgcaattgtgatataaaaaggtattccaaacagtacgtgctgaaaacc

SEQ ID NO: 34 - DNA, 5656 bp, synthetic construct
  source 1..5656: mol_type = other DNA; note = pDP03
agcggccgccaccgcggtggaggggaattcccatgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatcctctacgccggacgcatcgtggccggatcaattccgttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcacggaattgatccccctcgaattgacgcgttatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatggccgcgggattaaaagtcggggattggtgaacaaaaaggtgtttctctctttaagagaaatatcgttttgctaaacagttgatattgaggtatcattttatcgtaaaagacatttttgctcaacaattgcttgacggaaatcaacaaattttagcattttgtaaaaaagtcgctatataatttggtgaattggagttattttcatatttttgcatcccgaagagtttctcttaaagagagaaacatcttttgcataccttttccgaccgaatttttatgtcgtaaagaggggctttgcagggggtggactcagaaagatgagaatagatgactattgtagttgaaacacatagaaagttgctgatatacagaccgatacgcatatcgggatgaaccatgagtacgttcttttctcaaaaaacataaatattcgaaaagagatgcaataaattaaggagaggttataatgaacaaagtaaatataaaagatagtcaaaattttattacttcaaaatatcacatagaaaaaataatgaattgcataagtttagatgaaaaagataacatctttgaaataggtgcagggaaaggtcattttactgctggattggtaaagagatgtaattttgtaacggcgatagaaattgattctaaattatgtgaggtaactcgtaataagctcttaaattatcctaactatcaaatagtaaatgatgatatactgaaatttacatttcctagccacaatccatataaaatatttggcagcataccttacaacataagcacaaatataattcgaaaaattgtttttgaaagttcagccacaataagttatttaatagtggaatatggttttgctaaaatgttattagatacaaacagatcactagcattgctgttaatggcagaggtagatatttctatattagcaaaaattcctaggtattatttccatccaaaacctaaagtggatagcacattaattgtattaaaaagaaagccagcaaaaatggcatttaaagagagaaaaaaatatgaaacttttgtaatgaaatgggttaacaaagagtacgaaaaactgtttacaaaaaatcaatttaataaagctttaaaacatgcgagaatatatgatataaacaatattagtttcgaacaatttgtatcgctatttaatagttataaaatatttaacggctaaaaacaataggccacatgcaactgtaaatgtttacgcgtcctcggtaccgcttcttccacaacagtctgcggttcctgtactatcacaggttcatcttctcccggtgctgtcgcttcattctcttccacgaccttctcttctatcaccggacattcaacttcctctattacaggggcttgttcttcaacaatgggggcttcactttccgcttccgctacaggagaaggcgaggcttccaccggcacagcacttatctcttccgacaactgttccaaacgctcctgcatacgtaggatgctccgcttcaacagttcagacaaagtctgagtcggctctttagaaaacgtattcatgagtagcttcagctcatgaacatccagctcaatatctgttaatagcttctgtttcatttccatccattgttattatggtgcgaatgtaggaataatttatgcaataacttaaaataatggataatttaacagttcttgtacatcgaattgtttacctttgtccactcatttatttaatcaagaaagcacaaaaatcacatggtattattttcagaagaccacatacaagagaccagaagaagaggtagaatagaggtaatctgcggttcaatgttctccggaaagacggaggaattgattcgtaggatgaagagagccaagttcgcacgccagcgtgtagagatattcaaaccggccatcgacacccgttattcggaagaagacgtggtatcacacgacagtcattccatcgcgtccacccctatcgattcatcggctagtatcctgttgttcacttctgaaatagatgtagtcggcatcgacgaagcacagttctttgatgacggactgattgacgtatgcaggcagcttgccaataacggcatccgtgtcatcatcgccggactggatatggactttaaagggaatcccttcggaccgatgcctcaattatgtgccattgccgatgaagtttccaaagttcatgcgatctgcgtgaagtgcggtgatctggcatctttctctcatcgtaccgtcaagaatgacaagcaggtacttctaggagagacagccgaatacgagccgctttgcagggagtgctacctccgtgcacggggagaggacggacagaagatataaactctatttaaaacatatatggaaagaaagaagataacattcgagtcgacggtatcgataagcttgatatcgaattcctgcagcccggggtcagattgctatgggtgctgaccaggctcagactctgaaagctatccgtgaagctgaagcatatccgggaccatcactgatcatcgcttacgctccatgtatcaaccacggtctgaaagccggtatgggtaagagccaggaagaagaagaaaaggctgttaagtgcggttactggcacttgtggcgttacaaccctgctttggaagaagaaggcaagaatccgttccaactggatagcaaagaacctaactgggaagacttccaaggtttcttgaagggtgaagttcgttacgcttctgtaatgaaacaatatcctgctgaagctgaagaactgtttaaagctgctgaagaaaacgctaaatggcgttacaacagctacaaacgtcttgccagagaaaactggggagctgatacagctgaataataagtagaaatactttgtacataataaaaggcaggaatcctggaaacagattcctgccttttttgtttatatcttagaagtaacgattacttctcacgaagatattcattaaaactctaaagatatgatcggatttgttatttggattatcggtttgatactgactttcagagctgccatagagatttggaatgtaaatgcggctgtagagaaaagacttatagcgattgtattaatcgttcttaccagctggctgggactacttttctactacttctatggtaaagaaagaatggcgggatggttgaagtagtgtttgcatatcacatacaataaaatggtttaggttattaaaataatatacacagcaagacaagaacaaacgataacaggaatctttctttatatcgtataaagttctgatttaaataagggacaaataacaatgaatcccgaaataattaataaaatgattattgacagactgagtaattaaaaaaggaatagaatttgataagataatcctttcaacgaatagcaaaaaatagcatttttatgtataaaaacagcctgttccagtatcttgctggcacaaagatacaaaaaacggattaaacatacaaattatattcaaaatccggggcatacagagggctttatttgtaatgatatggaaagtttccgggtaatattcccctatcactcagcaaaatagaacaaaccggcagataaatataaaataagtttgaatatttcacttaaacaccgtatatttggcgtcaataactaaactagatttatatgttcaacagggctattacaattttcctggggaaggataattgtcattacacaacaaaagaaaaacaaacaggtactgccacactagacgacaaaagccaagtacagttccttagtaaaaagaaccccaaaatgccgtttttctatagccggcggaatctctacatgataggtttcctgctcgcttttatagtgactctgctcgaatttatacgaggacggcatcaaaactttatggtcttctcggacgctactcaattcttctggcaaggcat

SEQ ID NO: 35 - DNA, 116 bp, synthetic construct
  source 1..116: mol_type = other DNA; note = Inducible promoter PTetR56
acggttttgttttgtttgcaatggttaatctattgttaaaatttaaagtttcacttgaactttcaaataatgttcttatatttgcagtgtccctatcagtgatagagagtaggact

SEQ ID NO: 36 - DNA, 9268 bp, synthetic construct
  source 1..9268: mol_type = other DNA; note = PTetR56-opuA pNBU2 plasmid
tttatctatccattcagtttgatttttcaggactttacatcgtcctgaaagtatttgttggtaccgaggacgcgacagctgcagacgcgttatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatggccgcgggattaaaagtcggggattggtgaacaaaaaggtgtttctctctttaagagaaatatcgttttgctaaacagttgatattgaggtatcattttatcgtaaaagacatttttgctcaacaattgcttgacggaaatcaacaaattttagcattttgtaaaaaagtcgctatataatttggtgaattggagttattttcatatttttgcatcccgaagagtttctcttaaagagagaaacatcttttgcataccttttccgaccgaatttttatgtcgtaaagaggggctttgcagggggtggactcagaaagatgagaatagatgactattgtagttgaaacacatagaaagttgctgatatacagaccgatacgcatatcgggatgaaccatgagtacgttcttttctcaaaaaacataaatattcgaaaagagatgcaataaattaaggagaggttataatgaacaaagtaaatataaaagatagtcaaaattttattacttcaaaatatcacatagaaaaaataatgaattgcataagtttagatgaaaaagataacatctttgaaataggtgcagggaaaggtcattttactgctggattggtaaagagatgtaattttgtaacggcgatagaaattgattctaaattatgtgaggtaactcgtaataagctcttaaattatcctaactatcaaatagtaaatgatgatatactgaaatttacatttcctagccacaatccatataaaatatttggcagcataccttacaacataagcacaaatataattcgaaaaattgtttttgaaagttcagccacaataagttatttaatagtggaatatggttttgctaaaatgttattagatacaaacagatcactagcattgctgttaatggcagaggtagatatttctatattagcaaaaattcctaggtattatttccatccaaaacctaaagtggatagcacattaattgtattaaaaagaaagccagcaaaaatggcatttaaagagagaaaaaaatatgaaacttttgtaatgaaatgggttaacaaagagtacgaaaaactgtttacaaaaaatcaatttaataaagctttaaaacatgcgagaatatatgatataaacaatattagtttcgaacaatttgtatcgctatttaatagttataaaatatttaacggctaaaaacaataggccacatgcaactgtaaatgtttacgcgggtaccgacaccgcggtggaggggaattcccatgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatcctctacgccggacgcatcgtggccggatcaattccgttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcacggaattgatccccctcgaattggaattcgttcctcgattcagatctaaaggatccaatctagataaaacgaaaggctcagtcgaaagactgggcctttcgttttacaattgggctaccttttttttgttttgtttgcaatggttaatctattgttaaaatttaaagtttcacttgaactttcaaataatgttcttatatttccagtgtcgaaagaaacaaagtaggactgatcacacaacatttaaaaaataacattatgaaagcactcgaaaagagattcaaaggtaatattaacaataatttattttcaatgtccagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgagacgtaagtggagtgaccttaaatcaaggacgatattaatcggaaggagtattcaacgtgatgaagtcgcagggttgacgtgggacgtctctcggtataacgccggaaccgtcccccgctagaggcatcaaataaaacgaaaggctcagtcgaaagactgggcctttcgttttatctgttgtttgtcggtgaacgctctcctgagtaggacaaatgcggacaataaaggatcgtcagaaggtaacctgttatacaacgggcggacaataaaggatcgtcagaaggtaacctgttatacaacggttttgttttgtttgcaatggttaatctattgttaaaatttaaagtttcacttgaactttcaaataatgttcttatatttgcagtgtccctatcagtgatagagagtaggactgatcacacaacatttaaaaaataacattatgaaagcactcgaaaagagattcaaaggtaatattaacaataatttattttcaatgagtaaaatagaaattaaagatctctatctggtttttggcaatgagaagcagaaggctttgaagatgttgaaagaaggtaaaacaaaaagtgaaatattgaaagcgaccggatgtacagttgcggtgaaagatgcgaatctttccattaatgaaggcgaaatattcgtcattatggggctgtccggtagtggaaagtccactttactccgctgcatcaaccgtttgatacgccctacttccggagaagtgattatcaatggaacagacatagccaaagtgtcggataaggaactgttgcagattcgccggaaagaactggctatggtctttcagaacttcggtctgcttcctcaccgttctgtcttgcataatatagctttcgggctcgaactacagggagtaaaaaagggagaacgggagcagaaagcgatggaaagtatgcaactggtggggctgaaaggctacgagaatcagatggtaagtgaactgtcgggtgggatgcagcagcgtgtcgggctggcgcgggctttggccaataatcccgaagtcctgttgatggatgaggccttctcggcactcgatcctttgatccgtgtgcagatgcaggatgaactgctgactttgcagtcgaagatgaaaaaaacgattgtctttattactcacgacctgagtgaagcgattaaattgggtgaccggattgctatcatgaaggatggagaaattgtacaaatcggtacatcggaagaaatcctgaccgaacctgccaacgcttatgtagaacgatttgtggagaatgtagatcgaagcaagattataacagcttcctctgttatggtagataaacccatagtggcacgtttgaagaaagaaggcccggaggtgctgatccgtaagatgcgcgaacggaatctgaccgttcttccggtagtcgattccaataatctgttagtaggagaggtgcggttgaatgacttgctgaaactacgaaaagagcaaatccgctccatcgagtccgtagtaaggcatgaagttcattcagtgctgggtgatacggtgctggaagatattcttccgttgatgacaaaaacgaactcgccaatctgggtcgtaaatgagaatcgggagtttgaaggcgtagtgccactttcctcattgatcatcgaagtaacgggaaaagataaagaagaaattaatgaaatcattcaaaacgcaatagaattatgataaatatcggacaatatatagagactgccattaattggctcacagaacattttgcttccttttttgatgctctcagcatggggatcggaggattcatagacggttttcagcacgtactgtttggaatacctttttatatcacaattgcagtgctggctgcgctcgcctggtttaagtcgggtaaggggacagccgtatttaccctcttgggtttattgcttatttacggcatgggcttttgggaagaaacgatgcagacattggcattggtactttcttccacttgtctggcattgcttttaggcgttccgttgggaatctggactgccaacagtgatcgttgtaacaagataatgcgccctgtcctcgactttatgcagacaatgcctgcctttgtctatctgattcctgccgttctattcttcggattgggaactgtgccgggagcttttgccaccatcatctttgcaatgcctcctgtggttcgtctgaccggtctgggcattcgtcaggttcctaaaaacgtagtagaagcctcccgctcttttggagctactccctggcagcttctttataaggtgcaattgcctttggcgttgcctactattctgacgggaatcaaccagacgatcatgatgtcactttcgatggtagtgattgctgccatgatttctgccggaggattgggagaaatcgtgctgaaaggtatcacccaaatgaaaatcggactcggatttgaaggcggtatcgcagtcgtgatattggcgatcgtgctggaccgcatcactcagggtatggcagataacagaaaaagtaaaaagaaataagaatcacaccaaagaaaggaataatcaatgaataagataaaaatgattgcggcaatgtttattgccggcatactcttaaccttcgtttcctgcggagacaacggaaagagtaagaagataagtatcgcttatgccaactggtcggaaggtattgcaatgacaaaccttgccaaagcgatttttgaagatcaggggtacgatgtaaagttgctgaatgccgaccttgcacctatatttacctctatctcccgtaagaaagcagacgtatttatggatgtctggatgcctgtcactatggaagattatatgaaacaatacggggataaactcgaagttatcggggatatttatgatggtgcccgtatcgggctggtggttccggattacgttacgatcaactcaatcgaagaactgaacgcggaaaaggaacgtttctccggtcagattgtcggtattgatgccggagccggtattatgaaagctaccgatcaggccatcaaagactatggattggattataaactgatgacttcgagcggacctgccatgactgcttccctaaagaaagcgattgataagaaagactggattgtcgtgacgggatggacgcctcactggatgttcgaccgtttcaagctgaaagttcttcaggacccgaagctgatatatggaaatacggaatccattcataccatagcatggaaaggttttagcgagaaagacccttttgctgccgaacttttgggcaacattcgtctgacagatgcggaaatcagctccttaatgactgctctggaagaggcgcagacaacggaaagacaggctgcccgtcaatggatggaagaacatcaggaactggtgaacagttggattcctgagaaagattgaatcctagctgattagctcgagaaggccatcctgacggatggccttttttttgtatcgagacgactagtgtaattgcctatcttccagtgatggaacagcatttgtgcattggctgcaacaatcagccttacttgtgcctgttctatttccgaaccgaccgcttgtatgaatccatcaaaattcgttttctctatgttggattccttgttgctcatattgtgatgataatttctacaaatatagtcattggtaactatctatgaaactgtttgatacttttatagttgattaaacttgttcatggcatttgccttaatatcatccgctatgtcaatgtagggtttcatagctttgtagtcgctgtgtcccgtccatttcatgaccacctgtgccgggattccgagagccagcgcattgcagatgaatgtccttcttcctgcatgggtactgagcaaagcgtatttgggtgtgacttcatcaatacgttcatttcccttgtagtaggtttcccgtacaggctcgttgatttctgccagttcgcccagctctttcaggtaatcgttcatcttctggttgctgatgacgggcagagccatgtaattctcgaaatggatgtccttgtatttgtccagtatggctttgctgtatttgttcagttcaatcgtcaggctgtcggcagtcttgactgtggttatttcgatgtggtcggacttcacatcgcttcttttcagattgcgaacatccgaataccgcaaactcgtaaagcagcagaacaggaaaacatcacgcacacgttccaggtattgcttatccttgggtatctggtagtctttcagcttgttcagttcatcccaagtcaggaagattacttttttcgaggtggttttcagtttcggtttgaacgtatcgtatgcaatgttctgatgatgtcctttcttgaagctccagcgcaggaaccatttgaggaatcccatttgcttgccgatggtgctgtttctcatatccttggtgtcacgcaggaagttgacgtattcgttcaatccaaactcgttgaaatagttgaacgttgcatcctccttgaactctttgaggtggttcctcactgctgcaaatttttcataggtggatgccgtccagttattctggttaccgcactcttttacaaactcatcgaacacctcccaaaagctgacaggggcttcttccggctgttcttcgctggtgtctttcattctcatgttgaaagcttccttcaactgttgggtcgttggcatgacctcctgcacctcaaattccttgaaaatattctggatttcggcatagtatttcagcaagtccgtattgatttcggctgcactttgctttagcttgttggtacatccgctctttacccgctgcttatctgcatcccatttggctacgtcaatccggtagcccgttgtaaactcgatgcgttggctggcaaagatgacacgcatacggatgggtacgttctctacgattggcacaccgttctttttccggctctccaatgcaaaaatgatgttgcgcttgatattcataattgggtgcgtttgaaattctacacccaaatatacacccaattattgagatagcaaaagacatttagaaacatttacttttactctatattgtaatttacacttgattatcagtcgtttgcagtcttatgatattctgtgaaagtataagttcgagagcctgtctctccgcaaaaaacgctgaaaatcagcagattgcaaaacaaacaccctgttttacacccaagaatgtaaagtcggctgtttttgttttatttaagataatacaaccactacataataaaagagtagcgatattaaaagaatccgatgagaaaagactaata

SEQ ID NO: 37 - DNA, 25 bp, synthetic construct
  source 1..25: mol_type = other DNA; note = EM427 F Gibson primer for pNBU2 backbone
atcctagctgattagctcgagaagg

SEQ ID NO: 38 - DNA, 37 bp, synthetic construct
  source 1..37: mol_type = other DNA; note = EM315 R Gibson Primer for pNBU2 Backbone and PTetR56 amplification
tgaaaataaattattgttaatattacctttgaatctc

SEQ ID NO: 39 - DNA, 70 bp, synthetic construct
  source 1..70: mol_type = other DNA; note = EM316 F Gibson primer for PTetR56 amplification
gattcaaaggtaatattaacaataatttattttcaatgtccagattagataaaagtaaagtgattaacag

SEQ ID NO: 40 - DNA, 70 bp, synthetic construct
  source 1..70: mol_type = other DNA; note = JB221 F Gibson primer for opuA amplification
aaggtaatattaacaataatttattttcaatgagtaaaatagaaattaaagatctctatctggtttttgg